(19) United States
(12) Patent Application Publication
(10) Pub. No.: US 2012/0130129 A1
(43) Pub. Date: May 24, 2012

(54) Efficient Method for Preparing Functionalized Benzosuberenes

(75) Inventors: Kevin G. Pinney, Woodway, TX (US); Madhavi Sriram, Winston-Salem, NC (US); Clinton George, Hewitt, TX (US); Rajendra P. Tanpure, Waco, TX (US)

(73) Assignee: BAYLOR UNIVERSITY, Waco, TX (US)

(21) Appl. No.: 13/298,156

(22) Filed: Nov. 16, 2011

Related U.S. Application Data
(60) Provisional application No. 61/534,723, filed on Sep. 14, 2011.
(60) Provisional application No. 61/414,349, filed on Nov. 16, 2010.

(51) Int. Cl. C07C 213/00; C07C 45/00; C07C 41/01; C07C 201/12

(52) U.S. Cl. 564/428; 568/306; 568/314; 568/586

(57) ABSTRACT

The disclosed process can efficiently synthesize functionalized benzosuberenes. The process provides an improved method of production of benzosuberene and compounds containing a benzosuberene moiety, which is characterized by a ring closing methodology comprising reaction of a 5-phenylpentanoic acid with Eaton's reagent to form the benzosuberone. The process, optionally, further includes steps for adding a functional group at the ketone position.

### I. CROSS REFERENCE TO RELATED APPLICATIONS

This application claims the benefit of US. Provisional patent application No. 61/414,349, “Discovery and Development of Highly Potent Benzosuberene Anti-Cancer Agents,” filed 16 Nov. 2010, and U.S. provisional patent application No. 61/534,723, “Efficient Method for Preparing Functionalized Benzosuberenes,” filed 14 Sep. 2011, each of which is incorporated by reference in its entirety.

### II. INTRODUCTION

A. Field

The invention relates to an improved method for the production of benzosuberene compounds.

B. Background

The present invention relates to an improved method of production of benzosuberene and compounds containing a benzosuberene moiety.

The discovery of small-molecule anticancer agents that demonstrate nanomolar to subnanomolar cytotoxicity against human cancer cell lines is noteworthy, and such compounds have the potential to become drug candidates through an appropriately focused development program. The discovery of compounds that demonstrate picomolar cytotoxicity is even more exciting. In previous studies, a functionalized benzosuberene-based phenol 23 was identified as one such compound (see, e.g., U.S. Pat. No. 7,429,681). Its structure is reminiscent of both colchicine and combretastatin A-4 (CA4—FIG. 1), which are natural products that are potent inhibitors of tubulin assembly. Both compounds interact with tubulin at a small-molecule binding site named the colchicine site. The functionalized benzosuberene-based phenol 23 also bears structural similarity to a dihydronaphthalene analog (DHN—FIG. 1) that is strongly cytotoxic and inhibitory against tubulin assembly (Sriram, et al., Bioorg. Med. Chem. 2008, 16, 8161-8171).

In addition to its antiproliferative mechanism of action, both CA4 and DHN damage tumor vasculature. Preliminary studies with the benzosuberene phenol 23, in appropriate prodrug form, also suggest vascular damage as one component of its overall mechanism of action as an antitumor agent (U.S. provisional patent application U.S. Ser. No. 61/414,349, filed 16 Nov. 2010 and Pinney, et al., CPRIT Innovations in Cancer Prevention and Research Conference, Austin, Tex., 2010, Nov., 17-19, each incorporated herein by reference).

Small-molecule anticancer agents that target solid-tumor vasculature represent an important emerging area of research significance. Solid tumors require nutrients and oxygen provided by a network of vasculature, which has distinct morphological differences compared with a corresponding vascular network feeding normal healthy tissue. Tumor vasculature is highly disorganized with abnormal bulges, blind ends, and shunts. It is also characterized as leaky and discontinuous. It is these physiological dissimilarities that collectively offer a therapeutic advantage for the selective targeting and disruption of tumor vasculature through small-molecule drug intervention.

Tumor vasculature can be efficiently targeted through two distinct mechanistic pathways. Angiogenic inhibiting agents (AIAs) prevent new blood vessel growth. Vascular disrupting agents (VDAs), on the other hand, damage existing tumor vasculature through a series of cell-signaling pathways. One important class of small-molecule VDAs bind to tubulin heterodimers at the colchicine site and thereby potently inhibit tubulin assembly. The colchicine site is located on the β-subunit of the αβ-tubulin heterodimer, near the interface between the two subunits.

In the late 1970's, the combretastatins were discovered by Pettit and co-workers in the South African bush willow tree, Combreturn caffrum. Combretastatin A-1 (CA1) and combretastatin A-4 (CA4) are two of the most potent compounds isolated from C. caffrum, and both have pronounced biological activity as VDAs and as inhibitors of tubulin polymerization. In prodrug forms, both of these compounds, as well as the synthetic combretastatin amino-analog 7, are currently in human clinical trials.

The original synthetic methodology towards the benzosuberene molecular scaffold (described in U.S. Pat. No. 7,429,681) utilized a cyanogen azide ring expansion reaction. While reliable, the yield for this overall process is relatively low. The task of the present invention was therefore to find an improved method of production of benzosuberene containing compounds. Accordingly, the present invention relates to an improved method of production of benzosuberene and compounds containing a benzosuberene moiety, which is characterized by a ring closing methodology comprising reaction of a 5-phenylpentanoic acid with Eaton's reagent to form the benzosuberone.

### III. SUMMARY OF THE INVENTION

One aspect of the invention provides a process of making a compound of formula I,

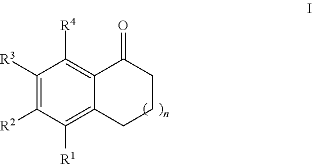

wherein
R¹, R², R³, and R⁴ are selected from the group consisting of hydrogen, halogen, C₁-C₆ alkyl, C₁-C₆ alkoxy, hydroxy, amino, nitro, and acylamino; and
n is an integer selected from 0, 1, 2 and 3;
   said process comprising reacting a benzaldehyde of formula A-1 with a Wittig reagent having the formula Ph₃P⁺(CH₂)₍ₙ₊₁₎COOH;

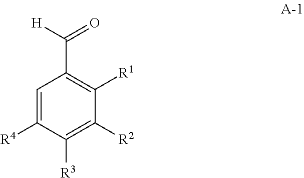

reducing the double bond formed in the Wittig reaction and effecting ring closure to form a compound of formula I. In certain implementations, n is 2. In another implementation, ring closure is effected with Eaton's reagent.

Another aspect of the invention provides a process of making a compound of formula II

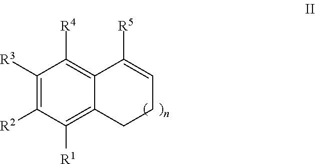

wherein
R¹, R², R³, and R⁴ are selected from the group consisting of hydrogen, halogen, C₁-C₆ alkyl, C₁-C₆ alkoxy, hydroxy, amino, and acylamino;
R⁵ is selected from the group consisting of unsubstituted aryl, substituted aryl, unsubstituted arylcarbonyl and substituted arylcarbonyl; and
n is an integer selected from 0, 1, 2 and 3;
   said process comprising reacting a benzaldehyde of formula A-1 with a Wittig reagent having the formula Ph₃P⁺(CH₂)₍ₙ₊₁₎COOH,

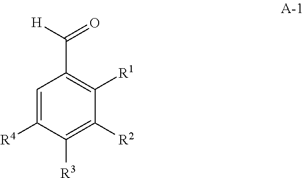

reducing the double bond formed in the Wittig reaction, effecting ring closure to form a benzoannulenone, and adding an R⁵ moiety at the ketone position of the benzoannulenone. In one implementation, R⁵ is a substituted or unsubstituted aryl, and adding the R⁵ moiety at the ketone position of the benzoannulenone comprises reacting the benzoannulenone directly with R⁵—Li to form a tertiary alcohol, and eliminating the tertiary alcohol to form the compound of formula II. In another implementation, R⁵ is a substituted or unsubstituted arylcarbonyl, and adding the R⁵ moiety at the ketone position of the benzoannulenone comprises converting the ketone of the benzoannulenone to a vinyl-lithium intermediate, reacting the intermediate with an electrophile to form an alcohol, and oxidizing the resultant alcohol to form the compound of Formula II.

### IV. BRIEF DESCRIPTION OF THE DRAWINGS

FIG. 1 illustrates the chemical structure of several tubulin binding agents.

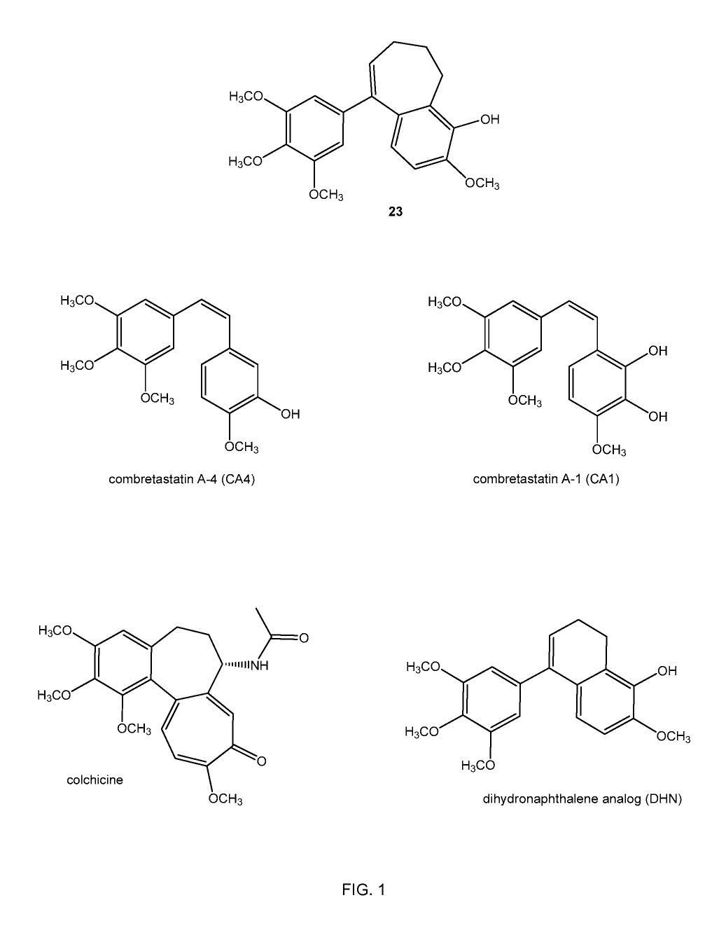

### V. DETAILED DESCRIPTION

### A. Overview

The disclosed process can efficiently synthesize functionalized benzosuberenes. The process provides an improved method of production of benzosuberene and compounds containing a benzosuberene moiety, which is characterized by a ring closing methodology comprising reaction of a 5-phenylpentanoic acid with Eaton's reagent to form the benzosuberone. The process, optionally, further includes steps for adding a functional group at the ketone position.

### B. Definitions

As used herein, the following definitions shall apply unless otherwise indicated.

“Alkyl” refers to monovalent saturated aliphatic hydrocarbyl groups having from 1 to 10 carbon atoms and preferably 1 to 6 carbon atoms. This term includes, by way of example, linear and branched hydrocarbyl groups such as methyl (CH₃—), ethyl (CH₃CH₂—), n-propyl (CH₃CH₂CH₂—), isopropyl ((CH₃)₂CH—), n-butyl (CH₃CH₂CH₂CH₂—), isobutyl ((CH₃)₂CHCH₂—), sec-butyl ((CH₃)(CH₃CH₂)CH—), t-butyl ((CH₃)₃C—), n-pentyl (CH₃CH₂CH₂CH₂CH₂—), and neopentyl ((CH₃)₃CCH₂—).

“Substituted alkyl” refers to an alkyl group having from 1 to 5 hydrogens replaced with substituents selected from the group consisting of alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, sulfonylamino, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO₃H, sulfonyl, sulfonyloxy, thioacyl, thiol, alkylthio, and substituted alkylthio, wherein said substituents are defined herein. In some embodiments, the alkyl has 1 to 3 of the aforementioned groups. In other embodiments, the alkyl has 1 to 2 of the aforementioned groups.

“Alkoxy” refers to the group —O-alkyl, wherein alkyl is as defined herein. Alkoxy includes, by way of example, methoxy, ethoxy, n-propoxy, isopropoxy, n-butoxy, t-butoxy, sec-butoxy, n-pentoxy, and the like.

“Substituted alkoxy” refers to alkoxy groups that are substituted with from 1 to 5 substituents selected from the group consisting of the same group of substituents defined for substituted alkyl. In some embodiments, the alkoxy has 1 to 3 of the aforementioned groups. In other embodiments, the alkoxy has 1 to 2 of the aforementioned groups.

“Acylamino” refers to the groups —NR²OC(O)alkyl, —NR²OC(O) substituted alkyl, N R²OC(O)cycloalkyl, —NR²OC(O) substituted cycloalkyl, —NR²OC(O)cycloalkenyl, —NR²OC(O) substituted cycloalkenyl, —NR²OC(O)alkenyl, —NR²OC(O) substituted alkenyl, —NR²OC(O)alkynyl, —NR²OC(O) substituted alkynyl, —NR²OC(O)aryl, —NR²OC(O) substituted aryl, —NR²OC(O)heteroaryl, —NR²OC(O) substituted heteroaryl, —NR²OC(O)heterocyclic, and —NR²OC(O) substituted heterocyclic, wherein R²⁰ is hydrogen or alkyl and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Amino” refers to the group —NH₂.

“Aryl” or “Ar” refers to a monovalent aromatic carbocyclic group of from 6 to 14 carbon atoms having a single ring (e.g., phenyl) or multiple condensed rings (e.g., naphthyl or anthryl) which condensed rings may or may not be aromatic (e.g., 2-benzoxazolinone, 2H-1,4-benzoxazin-3(4H)-one-7-yl, and the like), provided that the point of attachment is through an atom of the aromatic aryl group. Preferred aryl groups include phenyl and naphthyl.

“Substituted aryl” refers to aryl groups having 1 to 5 hydrogens replaced with substituents independently selected from the group consisting of alkyl, substituted alkyl, alkenyl, substituted alkenyl, alkynyl, substituted alkynyl, alkoxy, substituted alkoxy, acyl, acylamino, acyloxy, amino, substituted amino, aminocarbonyl, aminothiocarbonyl, aminocarbonylamino, aminothiocarbonylamino, aminocarbonyloxy, aminosulfonyl, aminosulfonyloxy, aminosulfonylamino, amidino, aryl, substituted aryl, aryloxy, substituted aryloxy, arylthio, substituted arylthio, carboxyl, carboxyl ester, (carboxyl ester)amino, (carboxyl ester)oxy, cyano, cycloalkyl, substituted cycloalkyl, cycloalkyloxy, substituted cycloalkyloxy, cycloalkylthio, substituted cycloalkylthio, cycloalkenyl, substituted cycloalkenyl, cycloalkenyloxy, substituted cycloalkenyloxy, cycloalkenylthio, substituted cycloalkenylthio, guanidino, substituted guanidino, halo, hydroxy, heteroaryl, substituted heteroaryl, heteroaryloxy, substituted heteroaryloxy, heteroarylthio, substituted heteroarylthio, heterocyclic, substituted heterocyclic, heterocyclyloxy, substituted heterocyclyloxy, heterocyclylthio, substituted heterocyclylthio, nitro, SO.sub.3H, sulfonyl, sulfonyloxy, thioacyl, thiol, alkylthio, and substituted alkylthio, wherein said substituents are as defined herein. In some embodiments, the aryl has 1 to 3 of the aforementioned groups. In other embodiments, the aryl has 1 to 2 of the aforementioned groups. In some embodiments, substituted aryl includes compounds containing oxo substituent in the non-aromatic ring fused to the aryl group. For example, 1-oxo-indan-4-yl, wherein the point of attachment is through the phenyl ring.

“Halo” or “halogen” refers to fluoro, chloro, bromo, and iodo and is preferably fluoro or chloro.

“Hydroxy” or “hydroxyl” refers to the group —OH.

“Heteroaryl” refers to an aromatic group of from 1 to 10 carbon atoms and 1 to 4 heteroatoms selected from the group consisting of oxygen, nitrogen, and sulfur within the ring. Such heteroaryl groups can have a single ring (e.g., pyridinyl, imidazolyl or furyl) or multiple condensed rings (e.g., indolizinyl, quinolinyl, benzimidazolyl or benzothienyl), wherein the condensed rings may or may not be aromatic and/or contain a heteroatom, provided that the point of attachment is through an atom of the aromatic heteroaryl group. In one embodiment, the nitrogen and/or sulfur ring atom(s) of the heteroaryl group are optionally oxidized to provide for the N-oxide (N→O), sulfinyl, or sulfonyl moieties. Preferred heteroaryls include pyridinyl, pyrrolyl, indolyl, thiophenyl, and furanyl.

“Nitro” refers to the group —NO₂.

Unless indicated otherwise, the nomenclature of substituents that are not explicitly defined herein are arrived at by naming the terminal portion of the functionality followed by the adjacent functionality toward the point of attachment. For example, the substituent “arylalkyloxycarbonyl” refers to the group (aryl)-(alkyl)-O—C(O)—.

It is understood that in all substituted groups defined above, polymers arrived at by defining substituents with further substituents to themselves (e.g., substituted aryl having a substituted aryl group as a substituent which is itself substituted with a substituted aryl group, which is further substituted by a substituted aryl group, etc.) are not intended for inclusion herein. In such cases, the maximum number of such substitutions is three. For example, serial substitutions of substituted aryl groups are limited to substituted aryl-(substituted aryl)-substituted aryl.

“Stereoisomer” and “stereoisomers” refer to compounds that have same atomic connectivity but different atomic arrangement in space. Stereoisomers include cis-trans isomers, E and Z isomers, enantiomers, and diastereomers.

Unless indicated otherwise, the nomenclature of substituents that are not explicitly defined herein are arrived at by naming the terminal portion of the functionality followed by the adjacent functionality toward the point of attachment. For example, the substituent “arylalkyloxycarbonyl” refers to the group (aryl)-(alkyl)-O—C(O)—.

Similarly, it is understood that the above definitions are not intended to include impermissible substitution patterns (e.g., methyl substituted with 5 fluoro groups). Such impermissible substitution patterns are easily recognized by a person having ordinary skill in the art.

### C. Methods of the Invention

The improved synthetic approach to the target benzosuberene analogs relies on a sequential Wittig reaction, selective reduction, cyclization strategy to assemble the requisite 6,7-ring fusion. Key features of this overall synthetic route include both improved yields and fewer reaction steps.

In one exemplary embodiment, the bicyclic compounds can be synthesized from substituted or unsubstituted benzaldehydes as illustrated in Scheme A. In scheme A, R¹, R², R³, and R⁴ are as defined herein.

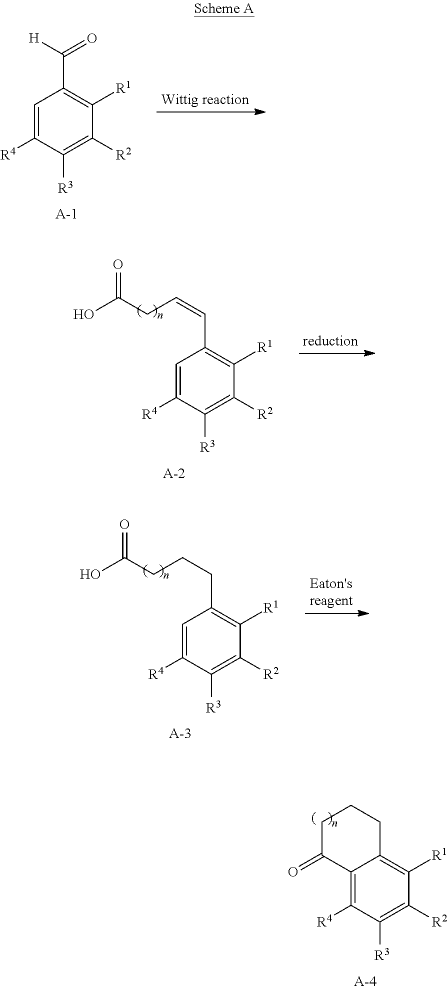

According to Scheme (I), a benzaldehyde A-1, optionally substituted at the 2-, 3-, 4- or 5-position, is alkenylated using an appropriate Wittig reagent under standard conditions to yield a phenylalkenoic acid A-2, e.g., 5-phenylpent-4-enoic acid. The double bond formed in the Wittig reaction is then reduced according to standard methods, such as catalytic hydrogenation with a platinum, palladium or nickel catalyst, to yield the corresponding phenylalkanoic acid A-3. Finally, the ring is closed in an acylation reaction in the presence of Eaton's reagent to benzoannulenone A-4.

Benzaldehydes A-1 and Wittig reagents can be purchased from commercial sources or, alternatively, can be synthesized using standard techniques. Skilled artisans will recognize that in some instances benzaldehyde A-1 may include functional groups that require protection during synthesis. The exact identity of any protecting group will depend upon the identity of the functional group being protected, and will be apparent to those of skill in the art. Guidance for selecting appropriate protecting groups, as well as synthetic strategies for their attachment and removal, can be found, for example, in Greene & Wuts, Protective Groups in Organic Synthesis, 4^{th} Edition, Wiley-Interscience (2006) and the references cited therein.

Thus, protecting group refers to a group of atoms that, when attached to a reactive functional group in a molecule, mask, reduce or prevent the reactivity of the functional group. typically, a protecting group may be selectively removed as desired during the course of a synthesis. Examples of protecting groups can be found in Greene and Wuts, as mentioned above, and additionally, in Harrison et al., Compendium of Synthetic Organic Methods, Vols. 1-12, 1971-2009. Representative hydroxyl-protecting groups include, but are not limited to, those where the hydroxyl group is either acylated to form acetate and benzoate esters or alkylated to form benzyl and trityl ethers, as well as alkyl ethers, tetrahydropyranyl ethers, trialkylsilyl ethers (e.g., TMS or TIPPS groups), sulfonyloxy, p-toluenesulfonyloxy and allyl ethers. Representative amino protecting groups include, but are not limited to, formyl, acetyl, trifluoroacetyl, benzyl, benzyloxycarbonyl (“CBZ”), tert-butoxycarbonyl (“Boc”), trimethylsilyl (“TMS”), 2-trimethylsilylethanesulfonyl (“TES”), trityl and substituted trityl groups, allyloxycarbonyl, 9-fluorenylmethyloxycarbonyl (“FMOC”), nitro-veratryloxycarbonyl (“NVOC”) and the like.

A specific embodiment of Scheme A for generating a benzosuberone utilizing (3-carboxypropyl)triphenylphosphonium bromide as the Wittig reagent is illustrated in Scheme A′ below:

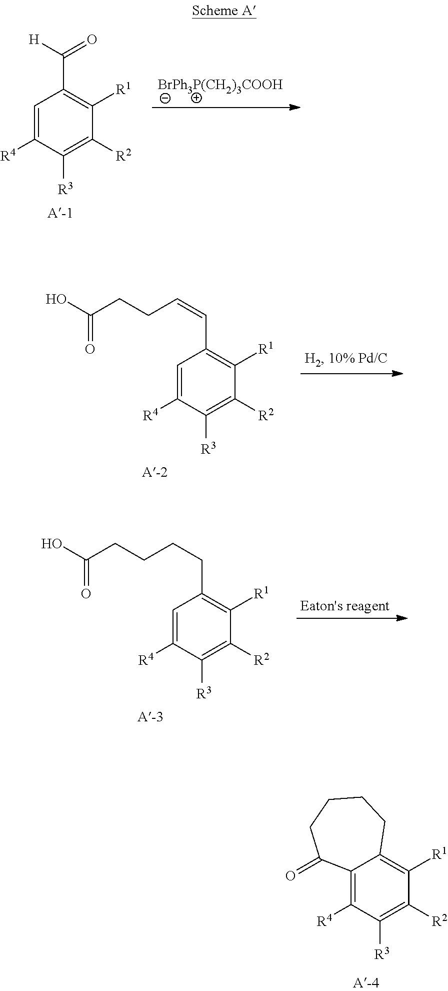

In a further embodiment, the benzoannulenone compounds synthesized in Schemes A and A′ can be used to generate aryl-substituted benzoannulenes, as illustrated in Scheme B.

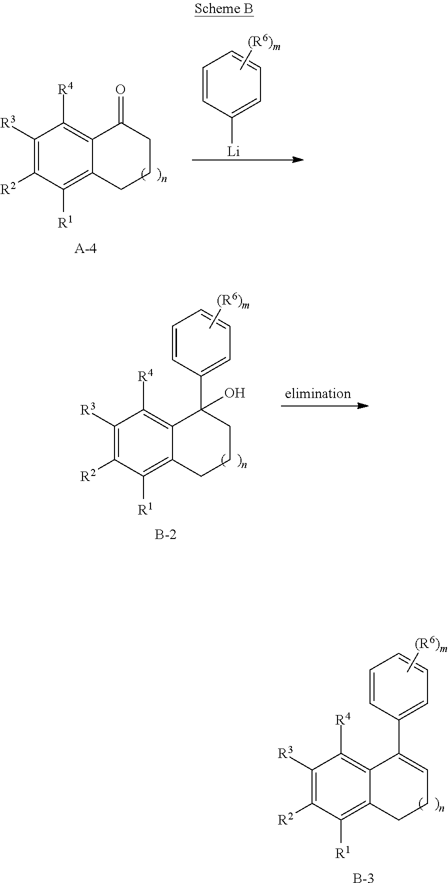

According to Scheme B, an optionally substituted aryllithium is reacted with the benzoannulenone. Alternatively, the aryllithium reagent can be replaced by an appropriate aryl Grignard reagent, which is commercially available or can be produced by generally known methods, to form the same tertiary alcohol compound. The resulting tertiary alcohol is eliminated according to standard methods known in the art. For example, the alcohol of B-2 can be eliminated by treatment with an appropriate acid, such as acetic acid (at reflux) or HCl (2M), or the like.

In a further embodiment, the benzoannulenone compounds synthesized in Schemes A and A′ can be used to generate arylcarbonyl-substituted benzoannulenes, as illustrated in Scheme C.

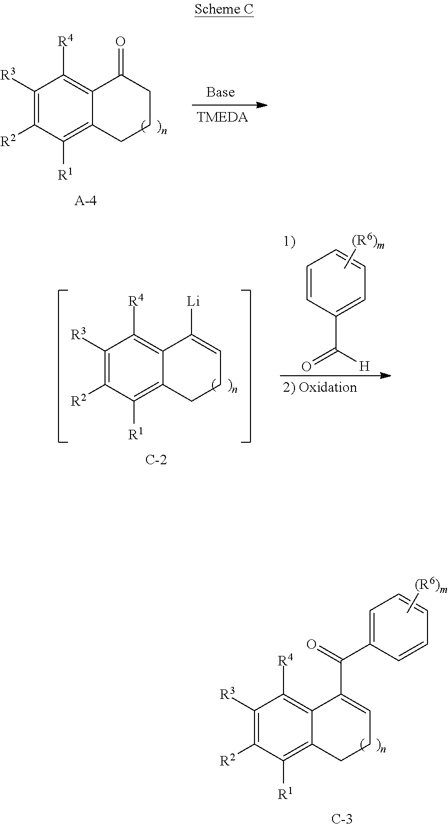

According to Scheme C, the ketone moiety of the benzoannulenone is converted to a vinyl-lithium intermediate (C-2) that is subsequently reacted with an appropriate electrophile. The resultant alcohol is then oxidized to the corresponding ketone (C-3). In an exemplary embodiment, the benzoannulenone is reacted with p-toluenesulfonylhydrazide to form the corresponding p-toluenesulfonylhydrazone analog, which upon treatment with an appropriate base, forms the vinyl-lithium intermediate. In the next step, an optionally substituted arylcarbaldehyde is reacted with C-2, to form the corresponding secondary alcohol intermediate. A strong base (such as n-BuLi in the presence of tetramethylethylenediamine (TMEDA or TEMED)) can be used to facilitate formation of the vinyl-lithium intermediate. The resultant hydroxyl can be oxidized, according to standard methods known in the art, to form the desired methanone (C-3).

In certain embodiments, phosphoric acid derivatives of the compounds of Formulae I or II can be formed by reacting the compound with POCl₃ and a base in the presence of a solvent to form the phosphoric acid. The term “base,” as used herein, means a Bronsted-Lowry base. A Bronsted-Lowry base is a reagent that is capable of accepting a proton (H+) from an acid present in a reaction mixture. Examples of Bronsted-Lowry bases include, but are not limited to, inorganic bases such as sodium carbonate, sodium bicarbonate, sodium hydroxide, potassium carbonate, potassium bicarbonate, potassium hydroxide, and cesium carbonate, organic bases such as triethylamine, diisopropylethylamine, diisopropylamine, cyclohexylamine, morpholine, pyrrolidone, piperidine, pyridine, 4-N,N-dimethylaminopyridine (DMAP), and imidazole. In a preferred embodiment, the base is an amine. In a particular embodiment, the base is triethylamine. Alternatively, phosphoric acid derivatives of the compounds of Formulae I and II can be formed by any other method known in the art.

The term “solvent,” as used herein, refers to a solvent that is inert to the ongoing reaction and sufficiently solubilizes the reactants to effect the desired reaction. Examples of suitable solvents include, but are not limited to, halogenated solvents, including, but not limited to, dichloromethane, chloroform, carbon tetrachloride, 1,2-dichloroethane, mixtures thereof, and the like. In a particular embodiment, the solvent is dichloromethane (DCM). Other examples of suitable solvents include ethers, such as diethyl ether, tetrahydrofuran (THF), and the like. In one exemplary embodiment, the solvent is tetrahydrofuran (THF).

Once the corresponding phosphoric acid is synthesized using POCl₃, the phosphoric acid can then be used in further reactions to synthesize various phosphoric acid salts and esters. A pharmaceutically acceptable salt of the phosphoric acid can be formed by further reacting the phosphoric acid with an appropriate amine or metal cation to form a pharmaceutically acceptable salt.

The end products of the reaction steps described herein can be isolated by conventional techniques, e.g. by extraction, crystallization, distillation, chromatography, etc. In one embodiment, any of the products of the reactions described herein can be processed through an additional upgrading step to remove unwanted impurities. The upgrading step can be performed in a number of different solvents, such as water and/or an alcohol (e.g., MeOH, EtOH, or IPA), followed by heating (20° C.-140° C., preferably 30° C.-100° C., preferably 35° C.-50° C.), for 10 minutes to 24 hours, preferably 15 minutes to 2 hours, preferably 60 minutes to 1.5 hours, and then followed by an isolation technique, such as filtering.

The invention is further understood by reference to the following examples, which are intended to be purely exemplary of the invention. The present invention is not limited in scope by the exemplified embodiments, which are intended as illustrations of single aspects of the invention only. Any methods that are functionally equivalent are within the scope of the invention. Various modifications of the invention in addition to those described herein will become apparent to those skilled in the art from the foregoing description and accompanying figures. Such modifications fall within the scope of the appended claims.

### VI. EXAMPLES

### A. Synthesis of Compounds

General Experimental Procedures. Methylene chloride (CH₂Cl₂), acetonitrile, and tetrahydrofuran (THF) were used in their anhydrous form as obtained from the chemical suppliers. Reactions were performed under an inert atmosphere using nitrogen gas unless specified. Thin-layer chromatography (TLC) plates (pre-coated glass plates with silica gel 60 F₂₅₄, 0.25 mm thickness) were used to monitor reactions. Purification of intermediates and products was carried out with a flash purification system using silica gel (200-400 mesh, 60 Å) or RP-18 prepacked columns. Intermediates and products synthesized were characterized on the basis of their ¹H NMR (500 MHz), ¹³C NMR (125 MHz), ¹⁹F NMR (470 MHz), ³¹P NMR (202 MHz), gHSQC, and gHMBC spectroscopic data. TMS was used as an internal standard for spectra recorded in CDCl₃. For spectra recorded in D₂O: δ¹H 4.80. All the chemical shifts are expressed in ppm (δ), coupling constants (J) are presented in Hz, and peak patterns are reported as broad (br), singlet (s), doublet (d), triplet (t), quartet (q), septet (sept), and multiplet (m). HRMS were obtained using electrospray ionization (ESI) technique. Purity of the final compounds was further analyzed at 25° C. using a HPLC system with a diode-array detector (λ=190-400 nm), a Zorbax XDB-C18 HPLC column (4.6 mm×150 mm, 5 μm), and a Zorbax reliance cartridge guard-column; eluents, solvent A, 0.025% ammonium trifluoroacetate in water, solvent B, acetonitrile; gradient, 90% A/10% B→40% A/60% B over 0 to 18 min; flow rate 1.0 mL/min; injection volume 20 μL; monitored at wavelengths (λλ254, 280 and 300 nm).

### 1. (Z/E) 5-(3′-Methoxy-2′-nitrophenyl)pent-4-enoic acid (1)

To a solution of anhydrous THF (60 mL) under N₂ was added (3-carboxypropyl)triphenylphosphonium bromide (2.66 g, 6.21 mmol) and potassium tert-butoxide (1.68 g, 14.9 mmol). The solution was allowed to stir for 1 h at ambient temperature. 3-Methoxy-2-nitrobenzaldehyde (1.07 g, 5.91 mmol) was added dropwise to the reaction mixture in THF (5 mL), which was allowed to stir for 12 h at ambient temperature. The solution was quenched with 2 M HCl (20 mL), and the organic solvent was evaporated under reduced pressure. The aqueous phase was extracted with EtOAc (4×30 mL), washed with brine, dried over Na₂SO₄, filtered, evaporated under reduced pressure, and purified by flash chromatography with a prepacked 100 g silica gel column [eluents; solvent A, EtOAc, solvent B, hexanes; gradient, 10% A/90% B→2% A/88% B (1 CV), 12% A/88% B→100% A/0% B (13 CV), 100% A/0% B (1.5 CV); flow rate, 40 mL/min; monitored at λλ254 and 280 nm]. Pentenoic acid analog 1 (0.879 g, 3.46 mmol, 58% yield) was obtained as a mixture of E and Z isomers and as a red solid, R_{f}=0.16 (50:50 hexanes:EtOAc).

¹H NMR (CDCl₃, 500 MHz) Reported as E & Z mixture: δ 7.38 (1H, t, J=8.1 Hz), 7.34 (1H, t, J=8.2 Hz), 7.12 (1H, dd, J=8.0 Hz, 0.8 Hz), 6.96 (1H, J=8.4 Hz), 6.89 (2H, d, J=8.4 Hz), 6.37 (1H, d, J=11.4 Hz), 6.31 (2H, m), 5.84 (1H, dt, J=11.4 Hz, 7.3 Hz), 3.90 (3H, s), 3.88 (3H, s), 2.54 (4H, m), 2.45 (4H, m). ¹³C NMR (CDCl₃, 125 MHz) Reported as E & Z mixture: δ 178.1, 178.0, 150.76, 150.74, 134.8, 134.3, 130.62, 130.60, 123.6, 123.5, 121.6, 118.0, 111.2, 110.9, 56.41, 56.39, 33.4, 33.1, 28.0, 23.8. HRMS, m/z: observed 252.0868 [M+H]⁺, (calcd for C₁₂H₁₄NO₅⁺, 252.0866).

### 2. 5-(3′-Methoxy-2′-nitrophenyl)pentanoic acid (2)

Pentenoic acid analog 1 (2.06 g, 8.21 mmol) under N₂ was added to anhydrous MeOH (100 mL) under N₂. To this solution was added 10% Pd/C (2.00 g) and stirred for 10 min at ambient temperature. 1,4-Cyclohexadiene (16.4 g, 205 mmol) was added to the mixture, which was stirred for 4 h. The reaction mixture was filtered through CELITE (diatomaceous earth), washed with EtOAc (4×25 mL), evaporated under reduced pressure, and purified by flash chromatography using 20% EtOAc/80% hexanes as eluent. Pentanoic acid analog 2 was obtained as a tan solid, (1.54 g, 6.10 mmol, 74% yield), R_{f}=0.22 (50:50, hexanes:EtOAc)

¹H NMR (CDCl₃, 500 MHz): δ 7.33 (1H, t, J=8.1 Hz), 6.87 (2H, dd, J=8.1 Hz, 3.7 Hz), 3.87 (3H, s), 2.58 (2H, m), 2.36 (2H, m), 1.67 (4H, m). ¹³C NMR (CDCl₃, 125 MHz): δ 179.4, 150.7, 141.8, 134.7, 130.7, 121.6, 110.1, 56.3, 33.6, 30.6, 29.7, 24.2. HRMS, m/z: observed 276.0845 [M+Na]⁺, (calc'd for C₁₂H₁₄NNaO₅⁺, 276.0842).

### 3. 1-Nitro-2-Methoxy-6,7,8,9-tetrahydro-5H-cyclobenzoheptan-5-one (3)

To a flask containing pentanoic acid analog 2 (0.281 g, 1.11 mmol) was added 5.5 mL of Eaton's reagent (7.7% P₂O₅ in CH₃SO₃H). The solid slowly dissolved with vigorous stirring and was allowed to stir for 12 h at ambient temperature. The solution was poured over ice, which was allowed to melt, then slowly neutralized with NaHCO₃ (aq.). The aqueous phase was extracted with EtOAc (4×25 mL). The combined organic extracts were washed with brine, dried over Na₂SO₄, filtered, evaporated under reduced pressure, and purified using flash chromatography with a prepacked 25 g silica column [eluents; solvent A, EtOAc, solvent B, hexanes; gradient, 5% A/95% B→7% A/93% B (1 CV), 7% A/93% B→60% A/40% B (13 CV), 60% A/40% B (1 CV); flow rate, 25 mL/min; monitored at λλ254 and 280 nm]. Ketone 3 (0.222 g, 0.945 mmol, 85% yield) was obtained as a white solid, R_{f}=0.34 (70:30 hexanes:EtOAc).

¹H NMR (CDCl₃, 500 MHz): δ 7.84 (1H, d, J=8.8 Hz), 6.97 (1H, d, J=8.8 Hz), 3.94 (3H, s), 2.79 (2H, m), 2.71 (2H, m), 1.90 (2H, m), 1.81 (2H, m). ¹³C NMR (CDCl₃, 125 MHz): δ 203.1, 153.3, 141.4, 134.0, 132.2, 131.9, 110.3, 56.6, 40.3, 26.2, 24.4, 20.2. HRMS, m/z: observed 236.0917 [M+H]⁺, (calc'd for C₁₂H₁₄NO₄⁺, 236.0917).

### 4. 1-Nitro-2-Methoxy-5-(3′,4′,5′-trimethoxyphenyl)-6,7,8,9-tetrahydro-5H-cyclobenzoheptan-5-ol (4)

A solution of 3,4,5-trimethoxyphenylbromide (0.389 g, 1.57 mmol) in anhydrous THF (15 mL) under N₂ was cooled to −78° C., then n-BuLi (0.57 mL, 2.5 M in hexanes) was added and stirred for 1 h. Ketone 3 (0.222 g, 0.945 mmol) was slowly added to the reaction mixture in anhydrous THF (3 mL) and allowed to warm to ambient temperature overnight with continuous stirring. On completion, the reaction mixture was quenched with H₂O (5 mL), and the solvent was evaporated under reduced pressure. The aqueous phase was extracted using EtOAc (4×15 mL). The combined organic extracts were washed with brine, dried over Na₂SO₄, filtered, evaporated under reduced pressure, and purified by flash chromatography using a prepacked 25 g silica column [eluents; solvent A, EtOAc, solvent B, hexanes; gradient, 5% A/95% B (1 CV), 5% A/95% B→12% A/88% B (1 CV), 12% A/88% B→100% A/0% B (13 CV), 100% A/0% B (2 CV); flow rate, 25 mL/min; monitored at λλ254 and 280 nm]. Tertiary alcohol 4 (0.320 g, 0.794 mmol, 84% yield) was obtained as a white solid, R_{f} 0.20 (50:50, hexanes: EtOAc).

¹H NMR (CDCl₃, 500 MHz): δ 7.68 (1H, d, J=8.8 Hz), 6.88 (1H, d, J=8.9 Hz), 6.46 (2H, s), 3.90 (3H, s), 3.85 (3H, s), 3.77 (6H, s), 3.10 (2H, m), 2.40 (1H, m), 2.19 (1H, s), 2.12 (1H, m), 1.94 (1H, m), 1.79 (2H, m), 1.54 (1H, m). ¹³C NMR (CDCl₃, 125 MHz): δ 153.3, 149.4, 142.4, 140.4, 138.5, 137.7, 133.5, 129.3, 109.2, 104.0, 79.7, 60.9, 56.2, 56.2, 40.9, 28.6, 26.3, 25.9. HRMS, m/z: observed 426.1525 [M+Na]⁺, (calc'd for C₂₁H₂₅NNaO₇⁺, 246.1523).

### 5. 1-Nitro-2-Methoxy-5-(3′,4′,5′-trimethoxyphenyl)-6,7,8,9-tetrahydro-5H-cyclobenzohepta-6-ene (5)

Tertiary alcohol 4 (0.266 g, 0.660 mmol) was dissolved in AcOH (10 mL) and refluxed at 140° C. for 3 h. The reaction was then cooled to ambient temperature and slowly neutralized with NaHCO₃ (aq.). The aqueous phase was then extracted with EtOAc (4×50 mL). The combined organic extracts were washed with brine, dried over Na₂SO₄, filtered, evaporated under reduced pressure, and purified by flash chromatography using a prepacked 25 g silica gel column [eluent; solvent A, EtOAc, solvent B, hexanes; gradient 5% A/95% B→7% A/93% B (1 CV), 7% A/93% B→60% A/40% B (10 CV), 60% A/40% B (2 CV); flow rate, 25 mL/min; monitored at λλ254 and 280 nm]. Nitro analog 5 (0.189 g, 0.491 mmol, 74% yield) was obtained as a white solid, R_{f}=0.29 (70:30, hexanes:EtOAc).

¹H NMR (CDCl₃, 500 MHz): δ 7.12 (1H, d, J=8.7 Hz), 6.87 (1H, d, J=8.5 Hz), 6.45 (2H, s), 6.43 (1H, t, J=7.4 Hz), 3.91 (3H, s), 3.87 (3H, s), 3.82 (6H, s), 2.56 (2H, t, J=6.9 Hz), 2.22 (2H, p, J=7.0 Hz), 2.00 (2H, q, J=7.3 Hz). ¹³C NMR (CDCl₃, 125 MHz): δ 153.1, 149.3, 141.5, 141.5, 137.7, 137.3, 134.9, 133.7, 131.8, 128.6, 109.7, 105.1, 60.9, 56.3, 56.2, 34.7, 27.3, 25.2. HRMS, m/z: observed 386.1599 [M+H]⁺, (calc'd for C₂₁H₂₄O₆⁺, 386.1598).

### 6. 2-Methoxy-5-(3′,4′,5′-trimethoxyphenyl)-6,7,8,9-tetrahydro-5H-cyclobenzohepta-6-ene-1-amine (6)

Nitro analog 5 (0.139 g, 0.361 mmol) was dissolved in AcOH (8 mL). Zinc dust (0.550 g, 8.42 mmol) was added to the solution, and the reaction mixture was stirred for 7 h at ambient temperature. The reaction was quenched with NaHCO₃ (aq.) and monitored by pH paper until a neutral pH was reached. The aqueous reaction mixture was extracted with EtOAc (4×25 mL), and the combined organic extracts were washed with brine, dried over sodium sulfate, and filtered. The solvent was evaporated under reduced pressure, and the crude product was purified by flash chromatography using a pre-packed 25 g silica gel column [eluent; solvent A, EtOAc, solvent B, hexanes; gradient 5% A/95% B→7% A/93% B (1 CV), 7% A/93% B→60% A/40% B (10 CV), 60% A/40% B (2 CV); flow rate, 25 mL/min; monitored at λλ254 and 280 nm]. The amino analog 6 (0.103 g, 0.290 mmol, 80% yield) was obtained as a yellow solid, R_{f}=0.47 (70:30, hexanes:EtOAc).

¹H NMR (CDCl₃, 500 MHz): δ 6.67 (1H, d, J=8.4 Hz), 6.52 (2H, s), 6.49 (1H, d, J=8.4 Hz), 6.30 (1H, t, J=7.4 Hz), 3.88 (3H, s), 3.86 (3H, s), 3.80 (6H, s), 2.59 (2H, t, J=6.9 Hz), 2.12 (2H, p, J=7.0 Hz), 1.95 (2H, q, J=7.2 Hz). ¹³C NMR (CDCl₃, 125 MHz): δ 152.8, 146.3, 143.5, 138.6, 137.2, 133.6, 132.4, 126.8, 126.3, 119.8, 107.6, 105.3, 60.9, 56.1, 55.6, 33.2, 25.6, 25.3. HRMS, m/z: observed 356.1857 [M+H]⁺, (calcd for C₂₁H₂₆O₄⁺, 356.1856).

### 7. (Z)/(E)-5-(2′,3′,4′-Trimethoxyphenyl)pent-4-enoic acid (7)

K-OtBu (12.30 g, 109.6 mmol) was added to a well-stirred solution of Wittig salt, (3-carboxypropyl)triphenylphosphonium bromide, (24.06 g, 56.05 mmol) in THF (400 mL, anhyd) at rt. The reaction mixture was then cooled to 0° C. and stirred for 15 mins. Solution of 2,3,4-trimethoxybenzaldehyde (9.84 g, 50.15 mmol) in THF (25 mL, anhyd) was added dropwise to the reaction mixture and the reaction was stirred until the temperature gradually warmed to rt. The reaction was quenched by careful addition of H₂O (50 mL) and extracted with Et₂O (2×200 mL). The aqueous phase was acidified with 2M HCl until the product precipitates making the solution cloudy and then becomes clear again. This acidified aqueous phase was extracted with EtOAc (3×100 mL). The combined organic phases were washed with brine, dried over Na₂SO₄, filtered and evaporated under reduced pressure. Flash chromatography of the crude using a prepacked 160 g silica column, Eluents; solvent A, EtOAc, solvent B, hexanes; gradient, 20% A/80% B over 3.18 min (1 CV), 20% A/80% B→60% A/40% B over 33.0 min (10 CV), 60% A/40% B over 6.36 min (2 CV); flow rate 50.0 mL/min; monitored at λλ254 and 280 nm afforded the mixture of E/Z-isomers 7 (9.48 g, 35.60 mmol, 64% yield), as a pale yellow liquid.

### 8. 5-(2′,3′,4′-Trimethoxyphenyl)pentanoic acid (8)

A suspension of 10% Pd/C and 7 (9.15 g, 34.4 mmol) in MeOH (200 mL) was stirred under H₂ gas (in balloons) for 24 h. The reaction was monitored for completion by filtering a little amount of the reaction mixture through CELITE (diatomaceous earth) and evaporating the solvent to record NMR data. On completion the reaction mixture was filtered through CELITE (diatomaceous earth) and the solvent was evaporated under reduced pressure to obtain a pale yellow liquid. Flash chromatography of the crude using a prepacked silica column afforded the pentanoic acid analog 8 (8.76 g, 32.64 mmol, 95% yield), as a pale yellow liquid.

¹H NMR (CDCl₃, 500 MHz): δ 6.81 (1H, d, J=8.5 Hz, H-6), 6.60 (1H, d, J=8.5 Hz, H-5), 3.87 (3H, s, OCH₃-2′), 3.86 (3H, s, OCH₃-3′), 3.84 (6H, s, OCH₃-4′), 2.57 (2H, t, CH₂-5), 2.39 (2H, t, CH₂-2), 1.68 (2H, m, CH₂-3), 1.61 (2H, m, CH₂-4). ¹³C NMR (CDCl₃, 125 MHz): δ 179.9 (C═O, C-1), 152.0 (C, C-4′), 151.8 (C, C-2′), 142.3 (C, C-3′), 128.0 (C, C-1′), 123.7 (CH, C-6′), 107.2 (CH, C-5′), 60.9 (CH₃, OCH₃-2′), 60.7 (CH₃, OCH₃-3′), 56.0 (CH₃, OCH₃-4′), 33.9 (CH, C-2′), 30.2 (CH, C-4), 29.3 (CH, C-4), 24.5 (C, C-2′).

### 9. 1,2,3-Trimethoxy-6,7,8,9-tetrahydro-5H-cyclobenzoheptan-5-one (9)

To pentanoic acid 8 (2.68 g, 10 mmol) in a flask, 40.2 mL of Eaton's reagent (10.64 g P₂O₅ in 100 mL methyl sulfonic acid) was added and the solution was stirred for 12 h under N₂. The reaction mixture was poured over ice and the ice was allowed to melt. The aqueous phase was extracted with CH₂Cl₂ (2×100 mL) and the combined organic phase was washed with NaHCO₃ (Satd. soln.) (2×200 mL). The organic phase was dried over Na₂SO₄ and solvent evaporated under reduced pressure to obtain ketone 9 as pale yellow liquid. Flash chromatography of the crude using a prepacked 50 g silica column, Eluents; solvent A, EtOAc, solvent B, hexanes; gradient, 10% A/90% B over 3.18 min (1 CV), 10% A/90% B→50% A/50% B over 33.0 min (10 CV), 50% A/50% B over 6.36 min (2 CV); flow rate 40.0 mL/min; monitored at λλ254 and 280 nm afforded ketone 9 (2.34 g, 9.34 mmol, 93% yield), as a pale yellow liquid.

¹H NMR (CDCl₃, 500 MHz): δ 7.13 (1H, s, H-4), 3.93 (3H, s, OCH₃-2), 3.88 (3H, s, OCH₃-3), 3.84 (6H, s, OCH₃-1), 2.94 (2H, dd, CH₂-9), 2.72 (2H, dd, CH₂-6), 1.83 (2H, m, CH₂-8), 1.81 (2H, m, CH₂-7). ¹³C NMR (CDCl₃, 125 MHz): δ 205.0 (C═O, C-5), 151.6 (C, C-3), 151.0 (C, C-1), 145.9 (C, C-2), 134.4 (C, C-4-a), 128.9 (C, C-1a), 107.5 (CH, C-4), 61.4 (CH₃, OCH₃-1), 60.8 (CH₃, OCH₃-2), 56.0 (CH₃, OCH₃-3), 40.8 (CH, C-6), 25.0 (CH, C-8), 23.0 (CH, C-9), 20.9 (C, C-7).

### 10. 1,2-Dihydroxy-3-methoxy-6,7,8,9-tetrahydro-5H-cyclobenzoheptan-5-one (10) and 2-Hydroxy-1,3-dimethoxy-6,7,8,9-tetrahydro-5H-cyclobenzoheptan-5-one (10′)

To a suspension of AlCl₃ (26.71 g, 101.4 mmol) in 200 mL CH₂Cl₂ cooled to 0° C., trimethylammonium chloride [TMAH] (9.55 g, 50.67 mmol) was added with stirring. The reaction mixture was allowed to warm to ambient temperature and stirred for 2 h. This clear yellow solution of ionic liquid was used as such for the deprotection of methyl ether of benzosuberone.

To a solution of (5.30 g, 21.2 mmol) of ketone 9 in CH₂Cl₂ (50 mL) cooled to 0° C., ionic liquid (36.00 mL, 23.32 mmol, 1.93M [TMAH][Al₂Cl₇] soln. in CH₂Cl₂) was added dropwise, while stirring the solution. The reaction was monitored by TLC. After the reaction was complete, it typically takes 5-10 min, ice cold water was added to quench the reaction. The mixture was stirred vigorously for 2 min and the organic layer was separated. Aqueous layer was extracted with CH₂Cl₂ (2×100 mL). Combined organic phase was washed with brine, and dried over MgSO₄, filtered and evaporated under reduced pressure. Flash chromatography of the crude using a prepacked 50 g silica column, Eluents; solvent A, EtOAc, solvent B, hexanes; gradient, 10% A/90% B over 3.18 min (1 CV), 10% A/90% B→40% A/60% B over 33.0 min (10 CV), 40% A/60% B over 6.36 min (2 CV); flow rate 40.0 mL/min; monitored at λλ254 and 280 nm; afforded catechol 10 (2.76 g, 12.4 mmol, 59%) and phenol 10′ (0.58 g, 2.45 mmol, 12%).

¹H NMR (500 MHz, CDCl₃) (catechol 10): δ 7.00 (1, s, H-4), 5.80 (1H, s, OH-2/1), 5.46 (1H, s, OH-1/2), 3.91 (3H, s, OCH₃-3), 2.98 (2H, s, CH₂-9), 2.72 (2H, s, CH₂-6), 1.81 (2H, s, CH₂-7, -8). ¹³C NMR (126 MHz, CDCl₃) δ 204.74, 144.64, 140.99, 135.67, 131.19, 123.42, 103.03, 56.18, 40.91, 24.64, 22.64, 21.19.

### 11. 1,2-Bis[(tert-butyldimethylsilyl)oxy]-3-methoxy-6,7,8,9-tetrahydro-5H-cyclobenzoheptan-5-one: (11)

To a solution of catechol 10 (2.76 g, 12.42 mmol), and DIPEA (5.67 g, 20.6 mmol) in anhydrous DMF (10 mL) at 0° C., TBSCI (5.65 g, 37.48 mmol) was added in portions while stirring. The reaction mixture was stirred for 6 h, quenched with H₂O (50 mL), and extracted with Et₂O (2×100 mL). The combined organic phase was washed with brine, dried over MgSO₄, filtered, and evaporated under reduced pressure. Flash chromatography of the crude product using a prepacked 50 g silica column [eluents; solvent A, EtOAc, solvent B, hexanes; gradient, 5% A/95% B over 1.39 min (1 CV), 5% A/95% B→30% A/70% B over 16.3 min (10 CV), 30% A/70% B over 5.18 min (2 CV); flow rate 40.0 mL/min; monitored at λλ254 and 280 nm] afforded bis-TBS protected analog 11 (5.25 g, 11.65 mmol, 94% yield) as a white solid.

¹H NMR (CDCl₃, 500 MHz): δ 7.00 (1H, s, H-4), 2.70 (2H, t, J=12.0 Hz, H-8), 3.80 (3H, s, OCH₃-3), 2.90 (2H, s, CH₂-5), 1.80 (4H, s, CH₂-6, -7), 1.02 (CH₃, (CH₃)₃), 1.02 (CH₃, (CH₃)₃), 0.19 (4-CH₃, Si(CH₃)₂). ¹³C NMR (CDCl₃, 125 MHz): δ 149.9 (C, C-3), 144.9 (C, C-1), 140.8 (C, C-2), 204.8 (C, C-9), 132.2 (C, C-10a), 128.9 (C, C-10b), 40.9 (CH, C-8), 104.9 (C, C-4), 54.9 (CH₃, OCH₃-3), 26.3 (CH₃, (CH₃)₃), 26.1 (CH₃, (CH₃)₃), 18.9 (C, (C(CH₃)₃), 18.6 (C, (C(CH₃)₃), −3.44 (CH₃, Si(CH₃)₂), −3.67 (CH₃, Si(CH₃)₂). Anal., Calcd for C₂₄H₄₂O₄Si₂: C, 63.95; H, 9.39; 0, 14.20; Si, 12.46. Found: C, 64.00; H, 9.44. HRMS, m/z: observed 451.2697 [M+1]⁺, (calcd for C₁₉H₃₁O₄Si₂⁺, 451.2694).

### 12. 1,2-Bis[(tert-butyldimethylsilyl)oxy]-3-methoxy-5-(3′,4′,5′-trimethoxyphenyl)-6,7,8,9-tetrahydro-5H-cyclobenzoheptan-5-ol (12)

To a solution of 3,4,5-trimethoxyphenylbromide (5.65 g, 22.9 mmol) in anhydrous THF (200 mL) at −78° C., n-BuLi (9.20 mL, 2.5 M) was added and the reaction stirred for 30 min. Benzosuberone 11 (5.15 g, 11.4 mmol) in 25 mL THF was added using a dropping funnel over a period of 15 min. The reaction mixture was stirred for overnight and allowed to warm to ambient temperature. On completion, the reaction mixture was quenched with H₂O (50 mL), and extracted with EtOAc (2×100 mL). The combined organic phase was washed with brine, dried over MgSO₄, filtered, and evaporated under reduced pressure. Flash chromatography of the crude product using a prepacked 100 g silica column [eluents; solvent A, EtOAc, solvent B, hexanes; gradient, 15% A/85% B over 1.39 min (1 CV), 15% A/65% B→50% A/50% B over 16.3 min (10 CV), 50% A/50% B over 5.18 min (2 CV); flow rate 40.0 mL/min; monitored at λλ254 and 280 nm] afforded alcohol 12 (6.65 g, 10.74 mmol, 94% yield) as a white solid.

Anal., Calcd for C₃₃H₅₄O₇Si₂: C, 64.04; H, 8.79; 0, 18.09; Si, 9.08. Found: C, 64.58; H, 8.71. HRMS, m/z: observed 641.3293 [M+Na]⁺, (calcd for C₃₃H₅₄O₇Si₂Na⁺, 641.3300).

### 13. 3-Methoxy-5-(3′,4′,5′-trimethoxyphenyl)-6,7,8,9-tetrahydro-5H-cyclobenzohepta-6-ene-1,2-diol (13)

A solution of alcohol 12 (6.55 g, 10.6 mmol) in AcOH (150 mL) and H₂O (100 mL) was heated to reflux at 110° C. for 12 h. The reaction mixture was cooled and the aqueous solvents evaporated over a rotavapor to obtain a crude product. Flash chromatography of the crude product using a prepacked 100 g silica column [eluents; solvent A, EtOAc, solvent B, hexanes; gradient, 20% A/80% B over 1.39 min (1 CV), 20% A/80% B→60% A/40% B over 16.3 min (10 CV), 60% A/40% B over 5.18 min (2 CV); flow rate 40.0 mL/min; monitored at λλ254 and 280 nm] afforded benzosuberene 13 (2.04 g, 5.48 mmol, 52% yield) as a white solid.

¹H NMR (CDCl₃, 500 MHz): δ 6.51 (2H, s, H-2′, H-6′), 6.37 (1H, t, J=7.4 Hz, H-8), 6.19 (1H, s, H-4), 3.87 (3H, s, OCH₃-4′), 3.81 (6H, s, OCH₃-3′, -5′), 3.73 (3H, s, OCH₃-3), 2.68 (2H, s, CH₂-9), 2.13 (2H, s, CH₂-8), 1.95 (2H, m, CH₂-7). ¹³C NMR (CDCl₃, 125 MHz): δ 152.9 (C, C-3′, C-5′), 144.4 (C, C-3), 142.8 (C, C-5), 140.9 (C, C-1), 138.2 (C, C-1′), 137.3 (CH, C-4′), 132.1 (CH, C-4-a), 131.2 (C, C-2), 127.8 (CH, C-6), 122.0 (C, C-1a), 105.1 (CH, C-2′, C-6′), 104.0 (C, C-4), 60.9 (CH₃, OCH₃-4′), 56.1 (CH₃, OCH₃-3′, -5′), 56.3 (CH₃, OCH₃-3). Anal., Calcd for C₂₁H₂₄O₆: C, 67.73; H, 6.50. Found: C, 67.46; H, 6.56.

### 14. 2-[(tert-Butyldimethylsilyl)oxy]-1,3-dimethoxy-6,7,8,9-tetrahydro-5H-cyclobenzoheptan-5-one (14)

To a solution of phenol 10′ (0.58 g, 2.45 mmol), and DIPEA (2.00 mL, 11.5 mmol) in anhydrous DMF (5 mL) at 0° C., TBSCI (0.82 g, 5.44 mmol) was added in portions while stirring. The reaction mixture was stirred for 6 h, quenched with H₂O (5 mL), and extracted with Et₂O (2×20 mL). The combined organic phase was washed with brine, dried over MgSO₄, filtered, and evaporated under reduced pressure. Flash chromatography of the crude product using a prepacked 25 g silica column [eluents; solvent A, EtOAc, solvent B, hexanes; gradient, 3% A/97% B over 1.39 min (1 CV), 3% A/97% B→30% A/70% B over 16.3 min (10 CV), 30% A/70% B over 5.18 min (2 CV); flow rate 25.0 mL/min; monitored at λλ254 and 280 nm] afforded ketone 14 (0.82 g, 2.34 mmol, 94% yield) as a white solid.

¹H NMR (CDCl₃, 500 MHz): δ 7.00 (1H, s, H-4), 3.80 (3H, s, OCH₃-3), 2.90 (2H, s, CH₂-5), 2.70 (2H, t, J=12.0 Hz, H-8), 1.80 (4H, s, CH₂-6, -7), 1.02 (CH₃, (CH₃)₃), 1.02 (CH₃, (CH₃)₃), 0.19 (4-CH₃, Si(CH₃)₂). ¹³C NMR (CDCl₃, 125 MHz): δ 204.8 (C, C-9), 149.9 (C, C-3), 144.9 (C, C-1), 140.8 (C, C-2), 132.2 (C, C-10a), 128.9 (C, C-10b), 104.9 (C, C-4), 54.9 (CH₃, OCH₃-3), 40.9 (CH, C-8), 26.3 (CH₃, (CH₃)₃), 26.1 (CH₃, (CH₃)₃), 18.9 (C, (C(CH₃)₃), 18.6 (C, (C(CH₃)₃), −3.44 (CH₃, Si(CH₃)₂), −3.67 (CH₃, Si(CH₃)₂). HRMS, m/z: observed 351.1887 [M+1]⁺, (calcd for C₁₉H₃₁O₄Si₂⁺, 351.1986).

### 15. 2-[(tert-Butyldimethylsilyl)oxy]-1,3-dimethoxy-5-(3′,4′,5′-trimethoxyphenyl)-6,7,8,9-tetrahydro-5H-cyclobenzoheptan-5-ol (15)

To a solution of 3,4,5-trimethoxyphenylbromide (1.04 g, 4.21 mmol) in anhydrous THF (50 mL) at −78° C., n-BuLi (1.70 mL, 2.5 M) was added and the reaction stirred for 30 min. Ketone 14 (0.73 g, 2.08 mmol) in 5 mL THF was added using a dropping funnel over a period of 15 min. The reaction mixture was stirred for overnight and allowed to warm to ambient temperature. On completion, the reaction mixture was quenched with H₂O (25 mL), and extracted with EtOAc (2×25 mL). The combined organic phase was washed with brine, dried over MgSO₄, filtered, and evaporated under reduced pressure. Flash chromatography of the crude product using a prepacked 25 g silica column [eluents; solvent A, EtOAc, solvent B, hexanes; gradient, 15% A/85% B over 1.39 min (1 CV), 15% A/85% B→50% A/50% B over 16.3 min (10 CV), 50% A/50% B over 5.18 min (2 CV); flow rate 25.0 mL/min; monitored at λλ254 and 280 nm] afforded alcohol 15 (0.80 g, 1.54 mmol, 74% yield) as a white solid.

### 16. 1,3-Dimethoxy-5-(3′,4′,5′-trimethoxyphenyl)-6,7,8,9-tetrahydro-5H-cyclobenzohepta-6-ene-2-ol (16)

A solution of 15 (0.77 g, 10.6 mmol) in AcOH (20 mL) and H₂O (20 mL) was heated to reflux at 110° C. for 24 h. The reaction mixture was cooled and the aqueous solvents evaporated over a rotavapor to obtain a crude product. Flash chromatography of the crude product using a prepacked 25 g silica column [eluents; solvent A, EtOAc, solvent B, hexanes; gradient, 10% A/40% B over 1.39 min (1 CV), 10% A/40% B→10% A/90% B over 16.3 min (10 CV), 40% A/60% B over 5.18 min (2 CV); flow rate 25.0 mL/min; monitored at λλ254 and 280 nm] afforded benzosuberene 16 (0.49 g, 5.48 mmol, 52% yield) as a white solid.

¹H NMR (CDCl₃, 500 MHz): δ 6.50 (2H, s, H-2′, H-6′), 6.38 (1H, s, H-4), 6.37 (1H, t, J=12.0 Hz, H-8), 5.62 (1H, s, OH-2), 3.92 (3H, s, OCH₃-1), 3.88 (3H, s, OCH₃-4′), 3.81 (6H, s, OCH₃-3′, -5′), 2.66 (2H, s, CH₂-5), 2.15 (2H, s, CH₂-6), 1.97 (2H, m, CH₂-7). ¹³C NMR (CDCl₃, 125 MHz): δ 152.9 (C, C-3′, C-5′), 145.2 (C, C-3), 144.5 (C, C-1), 137.6 (C, C-2), 142.9 (C, C-9), 138.0 (C, C-1′), 131.5 (C, C-10a), 137.4 (CH, C-4′), 128.3 (C, C-10b), 127.6 (CH, C-8), 108.1 (C, C-4), 105.2 (CH, C-2′, C-6′), 61.4 (CH₃, OCH₃-1), 60.9 (CH₃, OCH₃-4′), 56.2 (CH₃, OCH₃-3′, -5′), 56.4 (CH₃, OCH₃-3). Anal., Calcd for C₂₃H₂₆O₆: C, 68.38; H, 6.78; 0, 24.84. Found: C, 68.22; H, 6.85.

### 17. (Z)/(E)-5-(2′,3′-Dimethoxyphenyl)pent-4-enoic acid (17)

K-OtBu (11.3 g, 101.0 mmol) was added to a well-stirred solution of Wittig salt, (3-carboxypropyl)triphenylphosphonium bromide, (21.65 g, 50.43 mmol) in THF (500 mL, anhyd) at rt. The reaction mixture was then cooled to 0° C. and stirred for 15 mins. Solution of 2,3-dimethoxybenzaldehyde (8.42 g, 50.7 mmol) in THF (60 mL, anhyd) was added dropwise to the reaction mixture and the reaction was stirred until the temperature gradually warmed to rt. The reaction was quenched by careful addition of H₂O (50 mL) and extracted with Et₂O (2×200 mL). The aqueous phase was acidified with 2M HCl until the product precipitates making the solution cloudy and then becomes clear again. This acidified aqueous phase was extracted with EtOAc (3×100 mL). The combined organic phases were washed with brine, dried over Na₂SO₄, filtered and evaporated on a rotavapor. Flash chromatography of the crude using a prepacked 160 g silica column, Eluents; solvent A, EtOAc, solvent B, hexanes; gradient, 20% A/80% B over 3.18 min (1 CV), 10% A/80% B→60% A/40% B over 33.0 min (10 CV), 60% A/40% B over 6.36 min (2 CV); flow rate 50.0 mL/min; monitored at λλ254 and 280 nm afforded the mixture of E/Z-isomers 17 (6.98 g, 29.5 mmol, 58% yield), as a pale yellow liquid.

### 18. 5-(2′,3′-Dimethoxyphenyl)pentanoic acid (18)

A suspension of 10% Pd/C (0.70 g) and 17 (6.78 g, 28.7 mmol) in MeOH (100 mL) was stirred under H₂ gas (in balloons) for 24 h. The reaction was monitored for completion by filtering a little amount of the reaction mixture through CELITE (diatomaceous earth) and evaporating the solvent to record NMR data. On completion the reaction mixture was filtered through CELITE (diatomaceous earth) and the solvent was evaporated under reduced pressure to obtain a pale yellow liquid. Flash chromatography of the crude using a prepacked 50 g silica column, Eluents; solvent A, EtOAc, solvent B, hexanes; gradient, 10% A/90% B over 3.18 min (1 CV), 10% A/90% B→50% A/50% B over 33.0 min (10 CV), 50% A/50% B over 6.36 min (2 CV); flow rate 40.0 mL/min; monitored at λλ254 and 280 nm afforded the pentanoic acid analog 18 (6.71 g, 28.2 mmol, 98% yield), as a pale yellow liquid.

¹H NMR (CDCl₃, 500 MHz): δ 6.76 (1H, d, J=8.6 Hz, H-4, -6), 6.97 (1H, t, J=8.6 Hz, H-5), 3.85 (3H, s, OCH₃-3), 3.81 (3H, s, OCH₃-2), 2.38 (2H, s, CH₂-5), 2.65 (2H, t, J=12.0 Hz, H-2), 1.67 (2H, s, CH₂-3, -4). ¹³C NMR (CDCl₃, 125 MHz): δ 152.7 (C, C-3′), 147.1 (C, C-2′), 180.0 (C, C-1), 135.9 (C, C-1′), 121.9 (C, C-6′), 123.8 (C, C-5′), 110.2 (C, C-4′), 34.0 (CH, C-5), 30.1 (C, C-4), 24.5 (C, C-3), 60.6 (CH₃, OCH₃-2′), 55.7 (CH₃, OCH₃-3′).

### 19. 1,2-Dimethoxy-6,7,8,9-tetrahydro-5H-cyclobenzoheptan-5-one (19)

To pentanoic acid 18 (6.76 g, 28.4 mmol) in a flask, 75 mL of Eatons reagent (10.64 g P₂O₅ in 100 mL methyl sulfonic acid) was added and the solution was stirred for 12 h under N₂. The reaction mixture was poured over ice and the ice was allowed to melt. The aqueous phase was extracted with CH₂Cl₂ (2×100 mL) and the combined organic phase was washed with NaHCO₃ (Satd. soln.) (2×200 mL). The organic phase was dried over Na₂SO₄ and solvent evaporated under reduced pressure to obtain 19 as pale yellow liquid. Flash chromatography of the crude using a prepacked 100 g silica column, Eluents; solvent A, EtOAc, solvent B, hexanes; gradient, 10% A/90% B over 3.18 min (1 CV), 10% A/90% B→50% A/50% B over 33.0 min (10 CV), 50% A/50% B over 6.36 min (2 CV); flow rate 40.0 mL/min; monitored at λλ254 and 280 nm afforded the ketone 19 (5.39 g, 24.5 mmol, 86% yield), as a pale yellow liquid.

¹H NMR (CDCl₃, 500 MHz): δ 7.54 (1H, d, H-4), 6.84 (1H, d, H-3), 3.91 (3H, s, OCH₃-1), 3.80 (3H, s, OCH₃-2), 3.01 (2H, dd, CH₂-9), 2.70 (2H, dd, J=12.0 Hz, H-6), 1.85 (2H, s, CH₂-7), 1.81 (4H, s, CH₂-8). ¹³C NMR (CDCl₃, 125 MHz): δ 205.0 (C═O, C-1), 156.1 (C, C-1), 145.9 (C, C-2), 135.7 (CH, C-1a), 132.8 (C, C-4-a), 125.5 (C, C-4), 109.7 (C, C-3), 61.1 (CH₃, OCH₃-2), 55.8 (CH₃, OCH₃-2), 40.6 (CH, C-6), 24.9 (CH, C-7), 23.3 (CH, C-9), 20.9 (CH, C-8). Anal., Calcd for C₁₃H₁₆O₃: C, 70.89; H, 7.32; 0, 21.79. Found: C, 70.86; H, 7.39. HRMS, observed 221.1172 [M+1]⁺, (calcd for C₁₃H₁₇O₃⁺, 221.1172).

### 20. 1-Hydroxy-2-methoxy-6,7,8,9-tetrahydro-5H-cyclobenzoheptan-5-one (20)

A solution of ketone 19 (2.22 g, 10.1 mmol) in CH₂Cl₂ (5 mL) and ionic liquid (13.00 mL, 1.93M [TMAH][Al₂Cl₇] soln. in CH₂Cl₂) was heated to 80° C. in a microwave for 1 h. After the reaction was complete water was added to quench the reaction. The mixture was stirred vigorously for 2 min and the organic layer was separated. Aqueous layer was extracted with CH₂Cl₂ (2×25 mL). Combined organic phase was washed with brine, and dried over MgSO₄, filtered and evaporated under reduced pressure. Flash chromatography of the crude using a prepacked 100 g silica column, Eluents; solvent A, EtOAc, solvent B, hexanes; gradient, 10% A/90% B over 3.18 min (1 CV), 10% A/90% B→50% A/50% B over 33.0 min (10 CV), 50% A/50% B over 6.36 min (2 CV); flow rate 40.0 mL/min; monitored at λλ254 and 280 nm; afforded phenol 20 (1.59 g, 7.70 mmol, 77%) and catechol 59 (0.18 g, 0.94 mmol, 9%).

¹H NMR (CDCl₃, 500 MHz) (phenol 20): δ 7.34 (1H, d, J=8.6 Hz, H-4), 6.79 (1H, d, J=8.6 Hz, H-3), 3.94 (3H, s, OCH₃-2), 3.02 (3H, s, CH₂-5), 2.71 (2H, t, J=12.0 Hz, H-8), 1.85 (2H, s, CH₂-6), 1.80 (2H, s, CH₂-7). ¹³C NMR (CDCl₃, 125 MHz): δ 205.0 (C, C-5), 149.2 (C, C-2), 142.4 (C, C-1), 133.3 (CH, C-4-a), 127.7 (C, C-1a), 120.8 (C, C-4), 107.9 (C, C-3), 56.1 (CH₃, OCH₃-2), 40.8 (CH, C-6), 24.5 (CH, C-8), 23.1 (CH, C-9), 21.3 (CH, C-7). Anal., Calcd for C₁₂H₁₄O₃: C, 69.88; H, 6.84; 0, 23.27. Found: C, 69.93; H, 6.86. HRMS, m/z: observed 207.1016 [M+1]⁺, (calcd for C₁₂H₁₅O₃⁺, 207.1016).

### 21. 1-[(tert-Butyldimethylsilyl)oxy]-2-methoxy-6,7,8,9-tetrahydro-5H-cyclobenzoheptan-5-one (21)

To a solution of phenol 20 (6.36 g, 30.8 mmol), and DIPEA (5.75 g, 44.5 mmol) in anhydrous DMF (25 mL) at 0° C., TBSCI (7.01 g, 46.5 mmol) was added in portions while stirring. The reaction mixture was stirred for 6 h, quenched with H₂O (50 mL), and extracted with Et₂O (2×100 mL). The combined organic phase was washed with brine, dried over MgSO₄, filtered, and evaporated under reduced pressure. Flash chromatography of the crude product using a prepacked 50 g silica column [eluents; solvent A, EtOAc, solvent B, hexanes; gradient, 0% A/100% B over 1.39 min (1 CV), 0% A/100% B→30% A/70% B over 16.3 min (10 CV), 30% A/70% B over 5.18 min (2 CV); flow rate 40.0 mL/min; monitored at λλ254 and 280 nm] afforded aldehyde 21 (9.80 g, 30.6 mmol, 99% yield) as a white solid.

¹H NMR (CDCl₃, 500 MHz): δ 6.78 (1H, d, J=8.6 Hz, H-3), 7.38 (1H, d, J=8.6 Hz, H-4), 2.70 (1H, t, J=12.0 Hz, H-8), 3.84 (3H, s, OCH₃-2), 3.01 (2H, s, CH₂-5), 1.80 (2H, s, CH₂-6), 1.82 (2H, m, CH₂-7), 1.02 (CH₃, (CH₃)₃), 0.19 (CH₃, Si(CH₃)₂). ¹³C NMR (CDCl₃, 125 MHz): δ 153.2 (C, C-2), 141.8 (C, C-1), 205.3 (C, C-9), 133.x (CH, C-10a), 133.x (C, C-10b), 40.7 (CH, C-8), 122.3 (C, C-4), 108.8 (C, C-3), 54.8 (CH₃, OCH₃-2), 26.1 (CH₃, (CH₃)₃), 18.9 (C, (C(CH₃)₃), −3.90 (CH₃, Si(CH₃)₂). Anal., Calcd for C₁₈H₂₈O₃Si: C, 67.46; H, 8.81; 0, 14.98; Si, 8.76. Found: C, 67.70; H, 8.82. HRMS, m/z: observed 321.1881 [M+1]⁺, (calcd for C₁₈H₂₉O₃Si⁺, 321.1880).

### 22. 1-[(tert-Butyldimethylsilyl)oxy]-2-methoxy-5-(3′,4′,5′-trimethoxyphenyl)-6,7,8,9-tetrahydro-5H-cyclobenzoheptan-5-ol (22)

To a solution of 3,4,5-trimethoxyphenylbromide (16.8 g, 68.0 mmol) in anhydrous THF (400 mL) at −78° C., n-BuLi (27.2 mL, 2.5 M) was added and the reaction stirred for 30 min. Benzosuberone 21 (9.80 g, 30.6 mmol) in 25 mL THF was added using a dropping funnel over a period of 15 min. The reaction mixture was stirred for overnight and allowed to warm to ambient temperature. On completion, the reaction mixture was quenched with H₂O (50 mL), and extracted with EtOAc (2×100 mL). The combined organic phase was washed with brine, dried over MgSO₄, filtered, and evaporated under reduced pressure. Flash chromatography of the crude product using a prepacked 100 g silica column [eluents; solvent A, EtOAc, solvent B, hexanes; gradient, 5% A/95% B over 1.39 min (1 CV), 5% A/95% B→15% A/85% B over 16.3 min (10 CV), 15% A/85% B over 5.18 min (2 CV); flow rate 40.0 mL/min; monitored at λλ254 and 280 nm] afforded alcohol 22 (11.6 g, 17.4 mmol, 57% yield) as a white solid.

Anal., Calcd for C₂₇H₄₀O₆Si: C, 66.36; H, 8.25. Found: C, 66.07; H, 8.16.

### 23. 2-Methoxy-5-(3′,4′,5′-trimethoxyphenyl)-6,7,8,9-tetrahydro-5H-cyclobenzohepta-6-ene-1-ol (23)

A solution of 22 (11.6 g, 10.6 mmol) in AcOH (150 mL) and H₂O (100 mL) was heated to reflux at 110° C. for 12 h. The reaction mixture was cooled and the aqueous solvents evaporated over a rotavapor to obtain a crude product. Flash chromatography of the crude product using a prepacked 100 g silica column [eluents; solvent A, EtOAc, solvent B, hexanes; gradient, 5% A/95% B over 1.39 min (1 CV), 5% A/95% B→15% A/85% B over 16.3 min (10 CV), 15% A/85% B over 5.18 min (2 CV); flow rate 40.0 mL/min; monitored at λλ254 and 280 nm] afforded benzosuberene 23 (6.20 g, 17.4 mmol, 57% yield) as a white solid.

¹H NMR (CDCl₃, 500 MHz): δ 6.71 (1H, d, J=8.6 Hz, H-3), 6.57 (1H, d, J=8.6 Hz, H-4), 6.50 (2H, s, H-2′, H-6′), 6.34 (1H, t, J=12.0 Hz, H-8), 3.91 (3H, s, OCH₃-2), 3.86 (3H, s, OCH₃-4), 3.80 (6H, s, OCH₃-3′, -5′), 2.76 (2H, s, CH₂-5), 2.14 (2H, s, CH₂-6), 1.96 (2H, m, CH₂-7). ¹³C NMR (CDCl₃, 125 MHz): δ 152.8 (C, C-3′, C-5′), 145.0 (C, C-2), 142.8 (C, C-1), 142.8 (C, C-9), 138.5 (C, C-1′), 134.2 (CH, C-10a), 137.3 (CH, C-4′), 127.8 (C, C-10b), 127.2 (CH, C-8), 120.8 (C, C-4), 107.7 (C, C-3), 105.3 (CH, C-2′, C-6′), 60.9 (CH₃, OCH₃-4′), 56.1 (CH₃, OCH₃-3′, -5′), 56.0 (CH₃, OCH₃-2). Anal., Calcd for C₂₁H₂₄O₅: C, 70.77; H, 6.79; 0, 22.45. Found: C, 71.05; H, 6.77.

### 24. Disodium 2-Methoxy-5-(3′,4′,5′-trimethoxyphenyl)-6,7,8,9-tetrahydro-5H-cyclobenzohepta-6-ene-1-phosphate (24)

A solution of 23 (0.321 g, 0.83 mmol), POCl₃ (0.3 mL, 3.27 mmol), and pyridine (0.25 mL, 3.01 mmol) in DCM was stirred for 8 h. NaOH (5 mL, 2M) was added dropwise to the reaction mixture and the reaction was stirred for 5 min. The reaction mixture was extracted with DCM (2×25 mL). The combined organic phases were evaporated in vacuo. NaOH (5 mL, 2M) was added to the viscous liquid obtained and stirred at 60° C. for 15 min. The aqueous phase was evaporated in vacuo. Flash chromatography of the crude using a prepacked 25 g RP-18 silica column, Eluents; solvent A, Water, solvent B, ACN; gradient, 100% A/0% B over 1.19 min (1 CV), 100% A/0% B→60% A/40% B over 13.12 min (10 CV), 0% A/100% B over 3.57 min (3 CV); flow rate 25.0 mL/min; monitored at λλ254 and 280 nm afforded 24, (0.245 g, 0.478 mmol, 58% yield) as white solid.

### 25. 4-(2′,3′,4′-Trimethoxyphenyl)-4-hydroxybut-2-ynoic acid (25)

n-BuLi (2.5 M in hexanes, 41.0 mL, 102.5 mmol) was added dropwise to a well-stirred solution of prop-2-ynoic acid (3.65 g, 52.11 mmol) in THF (200 mL, anhyd) at −78° C. Solution of 2,3,4-trimethoxybenzaldehyde (8.65 g, 52.0 mmol) in THF (50 mL, anhyd) was added dropwise to the reaction mixture and the reaction was stirred until the temperature gradually warmed to rt. The reaction was quenched by careful addition of H₂O (50 mL) and extracted with Et₂O (2×100 mL). The aqueous phase was acidified with 2M HCl until the product precipitates making the solution cloudy and then becomes clear again. This acidified aqueous phase was extracted with EtOAc (3×100 mL). The combined organic phases were washed with brine, dried over Na₂SO₄, filtered and rotaevaporated. Flash chromatography of the crude using a prepacked silica column afforded the cinnamic acid 25 (7.41 g, 15.33 mmol, 76% yield), as a pale yellow liquid.

### 26. 4-(2,3-Dimethoxyphenyl)-4-hydroxybut-2-ynoic acid (26)

n-BuLi (2.5 M in hexanes, 130.0 mL, 325.00 mmol) was added dropwise to a well-stirred solution of prop-2-ynoic acid (9.54 g, 136.2 mmol) in THF (500 mL, anhyd) at −78° C. Solution of 2,3-dimethoxybenzaldehyde (25.01 g, 150.5 mmol) in THF (75 mL, anhyd) was added dropwise to the reaction mixture and the reaction was stirred until the temperature gradually warmed to rt. The reaction was quenched by careful addition of H₂O (50 mL) and extracted with Et₂O (2×200 mL). The aqueous phase was acidified with 2M HCl until the product precipitates making the solution cloudy and then becomes clear again. This acidified aqueous phase was extracted with EtOAc (3×250 mL). The combined organic phases were washed with brine, dried over Na₂SO₄, filtered and rotaevaporated. Flash chromatography of the crude using a prepacked silica column afforded the alcohol 26 (15.46 g, 65.44 mmol, 43% yield), as a pale yellow liquid.

### 27. 3-(2′,3′,4′-Trimethoxyphenyl)propanoic acid (27)

Through a suspension of 10% Pd/C (396 mg) and cinnamic acid 25 (4.82 g, 20.19 mmol) in MeOH (100 mL), H₂ gas (in balloons) was passed for 24 h. The reaction was monitored for completion by filtering a little amount of the reaction mixture through CELITE (diatomaceous earth) and evaporating the solvent to record NMR data. On completion, the reaction mixture was filtered through CELITE (diatomaceous earth) and the solvent was evaporated under reduced pressure to obtain propanoic acid analog 27 (4.73 g, 19.68 mmol, 98% yield), as a liquid.

¹H NMR (500 MHz, CDCl₃) δ 6.85 (1H, d, J=8.5 Hz, H-6), 6.59 (1H, d, J=8.5 Hz, H-5), 3.90 (3H, s, OCH₃-2′), 3.86 (3H, s, OCH₃-3′), 3.84 (3H, s, OCH₃-4′), 2.89 (2H, t, J=7.5, 8.0 Hz, H-3), 2.64 (2H, s, t, J=7.5, 8.0 Hz, H-2). ¹³C NMR (126 MHz, CDCl₃) δ 179.4 (C, C-1), 152.5 (C, C-4′), 151.9 (C, C-2′), 142.2 (C, C-3′), 126.1 (C, C-1′), 123.8 (CH, C-6′), 107.1 (CH, C-5′), 60.8 (CH₃, OCH₃-2′), 60.7 (CH₃, OCH₃-3′), 56.0 (CH₃, OCH₃-4′), 34.9 (CH₂, C-2), 25.2 (CH₂, C-3).

### 28. 4,5,6-Trimethoxy-2,3-dihydro-1H-inden-1-one (28)

To 27 (4.73 g, 19.68 mmol) in a flask, 40.0 mL of Eatons reagent (from Acros) was added and the solution was stirred for 12 h. The reaction mixture was poured over ice and the ice was allowed to melt. The aqueous phase was extracted with CH₂Cl₂ (2×100 mL) and the combined organic phase was washed with NaHCO₃ (Satd. soln.) (2×200 mL). The organic phase was dried over Na₂SO₄ and solvent evaporated under reduced pressure to obtain 28 as white solid. Flash chromatography of the crude using a prepacked 50 g silica column, Eluents; solvent A, EtOAc, solvent B, hexanes; gradient, 10% A/90% B over 3.18 min (1 CV), 10% A/90% B→50% A/50% B over 33.0 min (10 CV), 50% A/50% B over 6.36 min (2 CV); flow rate 40.0 mL/min; monitored at λλ254 and 280 nm afforded 28, (2.12 g, 11.90 mmol, 78% yield) as a white solid.

¹H NMR (500 MHz, CDCl₃) δ 7.02 (1H, s, H-7), 3.96 (3H, s, OCH₃-4), 3.95 (3H, s, OCH₃-5), 3.88 (3H, s, OCH₃-6), 3.04 (2H, t, J=5.7 Hz, H-3), 2.66 (2H, t, J=5.7 Hz, H-2). ¹³C NMR (126 MHz, CDCl₃) δ 206.0 (C, C-1), 154.2 (C, C-6), 150.0 (C, C-4), 147.6 (C, C-5), 141.6 (C, C-3a), 132.5 (CH, C-1a), 100.6 (CH, C-7), 61.1 (CH₃, OCH₃-5), 60.6 (CH₃, OCH₃-4), 56.2 (CH₃, OCH₃-6), 36.1 (CH₂, C-2), 22.4 (CH₂, C-3).

### 29. 2-Hydroxy-3-4-dimethoxybenzaldehyde (29) and 2,3-Dihydroxy-4-methoxybenzaldehyde (29′)

2,3,4-Trimethoxybenzaldehyde (5.00 g, 25.5 mmol) was dissolved in dry CH₂Cl₂ (15 mL) at 0° C. under nitrogen. Anhydrous BCl₃ (28.0 mL, 1.0 M soln in CH₂Cl₂) was added dropwise from a dropping funnel, and the reaction mixture stirred for 5 h. The reaction was quenched with H₂O (10 mL), the organic phase was separated, and the aqueous phase extracted with CH₂Cl₂ (2×25 mL). The combined organic phases were washed with brine, dried over Na₂SO₄, filtered, and concentrated to dryness under reduced pressure. Aldehyde 29 (3.75 g, 20.6 mmol, 81% yield) was obtained as a white powder and catechol aldehyde 29′ (0.20 g, 1.2 mmol, 5% yield) was obtained as a off-white powder

¹H NMR (500 MHz, CDCl₃) δ 11.2 (1H, s, OH-2), 9.75 (1H, s, CHO-1a), 7.29 (1H, d, J=9.0 Hz, H-6), 6.61 (1H, d, J=9.0 Hz, H-5), 3.95 (3H, s, OCH₃-4), 3.91 (3H, s, OCH₃-3). ¹³C NMR (126 MHz, CDCl₃) δ 194.9 (CH, C-1a), 159.4 (C, C-4), 155.7 (C, C-2), 136.2 (C, C-3), 130.2 (CH, C-6), 116.6 (C, C-1), 104.0 (CH, C-5), 60.8 (CH₃, OCH₃-3), 56.3 (CH₃, OCH₃-4).

### 30. 2-Tosyloxy-3,4-dimethoxybenzyldehyde (30)

To a stirred solution of aldehyde 29 (2.0 g, 11.0 mmol), DIPEA (4.0 mL, 23.0 mmol) in anhydrous DMF (10 mL) at rt, p-TsCI (4.18 g, 22.0 mmol) was added in portions. The reaction mixture was stirred 12 hrs and quenched with H₂O (10 mL) and the solution was extracted with CH₂Cl₂ (3×25 mL). The combined organic phases were washed with brine, dried over MgSO₄, filtered, and evaporated under reduced pressure. The crude product was subjected to flash column chromatography to afford aldehyde 30 (3.50 g, 10.4 mmol, 95% yield) as a white solid.

¹H NMR (500 MHz, CDCl₃) δ 9.86 (1H, s, CHO-1a), 7.86 (2H, d, J=8.35 Hz, H-2′, -6′), 7.68 (1H, d, J=8.8 Hz, H-6), 7.37 (2H, d, J=8.35 Hz, H-3′, -5′), 6.94 (1H, d, J=8.8 Hz, H-5), 3.94 (3H, s, OCH₃-4), 3.74 (3H, s, OCH₃-3), 2.48 (3H, s, CH₃-4′). ¹³C NMR (126 MHz, CDCl₃) δ 187.0 (CH, C-1a), 158.9 (C, C-4), 145.8 (C, C-2), 145.1 (C, C-3), 142.3 (C, C-3), 132.8 (C, C-1′), 129.9 (CH, C-3′,-5′), 128.4 (C, C-2′,-6′), 124.0 (C, C-1), 123.9 (CH, C-6), 110.6 (CH, C-5), 61.0 (CH₃, OCH₃-3), 56.4 (CH₃, OCH₃-4), 21.8 (CH₃, CH₃-4′).

### 31. (Z)/(E)-5-(3′,4′-dimethoxy-2′-(tosyloxy)phenyl)pent-4-enoic acid (31)

n-BuLi (2.5 M in hexanes, 5.4 mL) was added dropwise to a well-stirred solution of Wittig salt, (3-carboxypropyl)triphenylphosphonium bromide, (3.82 g, 8.90 mmol) in anhydrous THF (200 mL) at −50° C. The reaction mixture was then warmed to rt, stirred for 15 mins, and then cooled to −78° C. Aldehyde 30 (2.01 g, 5.97 mmol) dissolved in anhydrous THF (15 mL) was added dropwise to the reaction mixture and the reaction was stirred until the temperature gradually rose to rt. The reaction was quenched by careful addition of H₂O (50 mL) and the aqueous phase was extracted with Et₂OAc (3×200 mL), the combined organic phases were washed with brine. After drying with MgSO₄, the solvents were removed under reduced pressure and the crude product obtained was subjected to column chromatography to obtain the mixture of E/Z-isomers 31 (1.03 g, 2.53 mmol, 42% yield), as an off-white solid.

### 32. 5-(2′-Tosyloxy-3′,4′-dimethoxyphenyl)pentan-1-oic acid (32)

Through a suspension of 10% Pd/C (400 mg) and pentanoic acid 31 (1.25 g, 20.19 mmol) in MeOH (40 mL) and EtOH (15 mL), H₂ gas (in balloons) was passed for 12 h. The reaction was monitored for completion by filtering a little amount of the reaction mixture through CELITE (diatomaceous earth) and evaporating the solvent to record NMR data. On completion, the reaction mixture was filtered through CELITE (diatomaceous earth) and the solvent was evaporated under reduced pressure to obtain 32 (0.94 g, 2.3 mmol, 75% yield), as an off-white solid.

¹H NMR (500 MHz, CDCl₃): δ 7.93 (2H, d, J=8.2 Hz, H-2′, -6′), 7.35 (2H, d, J=8.2 Hz, H-3′, -5′), 6.89 (1H, d, J=8.6 Hz, H-6), 6.77 (1H, d, J=8.6 Hz, H-5), 3.82 (3H, s, OCH₃-4), 3.51 (3H, s, OCH₃-3), 2.58 (2H, t, H-5), 2.46 (3H, s, CH₃-4″), 2.34 (2H, m, H-2), 1.61 (4H, m, H-3,-4). ¹³C NMR (126 MHz, CDCl₃): δ 179.0 (C, C-1), 151.8 (C, C-4′), 144.7 (C, C-4″), 142.3 (C, C-2′), 142.1 (C, C-3′), 134.9 (C, C-1″), 129.5 (CH, C-3″,-5″), 129.0 (C, C-1′), 128.1 (C, C-2″,-6″), 123.9 (CH, C-6′), 110.8 (CH, C-5′), 60.5 (CH₃, OCH₃-3′), 56.1 (CH₃, OCH₃-4′), 33.7 (CH₂, C-2), 29.6 (CH₂, C-5), 29.5 (CH₂, C-4), 24.4 (CH₂, C-3), 21.7 (CH₃, CH₃-4″).

### 33. 1-Tosyloxy-2,3-dimethoxy-6,7,8,9-tetrahydro-5H-cyclobenzoheptan-5-one (33)

To pentanoic acid 32 (0.90 g, 2.2 mmol) in a flask, 14 mL of Eatons reagent (from Acros) was added and the solution was stirred for 12 h. The reaction mixture was poured over ice and the ice was allowed to melt. The aqueous phase was extracted with CH₂Cl₂ (3×100 mL) and NaHCO₃ powder was added in small amounts until the pH was neutral. The organic phase was treated with brine, dried over Na₂SO₄, filtered and solvent evaporated under reduced pressure to obtain 33 as white solid. Flash chromatography of the crude using a prepacked 25 g silica column, Eluents; solvent A, EtOAc, solvent B, hexanes; gradient, 25% A/75% B over 3.18 min (1 CV), 25% A/75% B→60% A/40% B over 33.0 min (10 CV), 60% A/40% B over 6.36 min (2 CV); flow rate 25.0 mL/min; monitored at λλ254 and 280 nm afforded 33, (0.70 g, 1.8 mmol, 81% yield) as a white solid.

¹H NMR (500 MHz, CDCl₃) δ 7.93 (2H, d, J=8.0 Hz, H-2′, -6′), 7.37 (2H, d, J=8.0 Hz, H-3′, -5′), 7.29 (1H, s, H-4), 3.87 (3H, s, OCH₃-2), 3.58 (3H, s, OCH₃-3), 2.95 (2H, t, J=5.3, 6.4 Hz, H-9), 2.73 (3H, t, J=5.3, 6.4 Hz, CH₃-6), 1.84 (2H, m, H-8), 1.81 (2H, m, H-7). ¹³C NMR (126 MHz, CDCl₃) δ 204.0 (C, C-5), 151.3 (C, C-3), 145.4 (C, C-2), 145.0 (C, C-4′), 141.2 (C, C-1), 134.32 (C, C-4-a), 134.28 (CH, C-1′), 129.9 (C, C-1a), 129.5 (CH, C-3′,-5′), 128.2 (C, C-2′,-6′), 110.9 (CH, C-4), 60.5 (CH₃, OCH₃-3), 56.0 (CH₃, OCH₃-2), 40.7 (CH₂, C-6), 24.7 (CH₂, C-8), 24.5 (CH₂, C-9), 21.7 (CH₃, CH₃-4′), 20.8 (CH₂, C-7).

### 34. 1-Tosyloxy-2,3-dimethoxy-5-(3′,4′,5′-trimethoxyphenyl)-6,7,8,9-tetrahydro-5H-cyclobenzoheptan-5-ol (34)

To a solution of 3,4,5-trimethoxyphenylbromide (0.85 g, 3.4 mmol) in anhydrous THF (100 mL) at −78° C., n-BuLi (1.4 mL, 2.5 M in hexanes) was added and the reaction stirred for 30 min. Benzosuberone 33 (0.67 g, 1.7 mmol) in 15 mL THF was added using a dropping funnel over a period of 15 min. The reaction mixture was stirred overnight and allowed to warm to ambient temperature. On completion, the reaction mixture was quenched with H₂O (100 mL), and extracted with Et₂O (150 mL) and EtOAc (15 mL). The combined organic phase was washed with brine, dried over MgSO₄, filtered, and evaporated under reduced pressure. Flash chromatography of the crude product using a prepacked 25 g silica column [eluents; solvent A, EtOAc, solvent B, hexanes; gradient, 25% A/75% B over 1.39 min (1 CV), 25% A/75% B 80% A/20% B over 16.3 min (10 CV), 80% A/20% B over 5.18 min (2 CV); flow rate 25.0 mL/min; monitored at λλ254 and 280 nm] afforded alcohol 34 (0.61 g, 1.1 mmol, 64% yield) as a white solid.

¹H NMR (CDCl₃, 500 MHz): δ 7.91 (2H, d, J=8.3 Hz, H-2″, -6″), 7.35 (2H, d, J=8.3 Hz, H-3″, -5″), 7.37 (1H, s, H-4), 6.46 (2H, s, H-2′, H-6′), 3.844 (3H, s, OCH₃-4′), 3.840 (3H, s, OCH₃-3), 3.78 (6H, s, OCH₃-3′, -5′), 3.54 (3H, s, OCH₃-2), 3.12 (1H, s, CH₂-9), 2.62 (1H, m, CH₂-6), 2.46 (3H, s, OCH₃-4″), 2.22 (1H, s, CH₂-9), 2.13 (1H, m, CH₂-6), 2.23 (1H, s, CH₂-9), 1.88 (1H, s, CH₂-718), 1.72 (2H, m, CH₂-7, -8), 1.72 (1H, m, CH₂-817). ¹³C NMR (CDCl₃, 125 MHz): δ 153.2 (C, C-3′, C-5′), 150.5 (C, C-3), 144.7 (C, C-4″), 141.7 (C, C-1), 141.4 (C, C-4-a), 140.7 (C, C-2), 139.9 (C, C-1′), 137.5 (CH, C-4′), 134.7 (C, C-1″), 129.4 (C, C-3″, C-5″), 128.4 (C, C-1a), 128.1 (C, C-2″, C-6″), 110.2 (CH, C-4), 104.2 (CH, C-2′, C-6′), 80.2 (C, C-5), 60.8 (CH₃, OCH₃-4′), 60.5 (CH₃, OCH₃-2), 56.2 (CH₃, OCH₃-3′, -5′), 56.0 (CH₃, OCH₃-3), 41.1 (CH₂, CH₂-6), 26.7 (CH₂, CH₂-718), 26.6 (CH₂, CH₂-9), 26.3 (CH₂, CH₂-718), 21.7 (CH₃, CH₃-4″).

### 35. 1-Tosyloxy-2,3-dimethoxy-5-(3′,4′,5′-trimethoxyphenyl)-6,7,8,9-tetrahydro-5H-cyclobenzoheptan-5-ene (35)

A solution of alcohol 34 (0.54 g, 0.97 mmol) in AcOH (20 mL) and H₂O (30 mL) was heated to reflux at 180° C. for 12 h. The reaction mixture was cooled and the aqueous solvents evaporated over a reduced pressure to obtain a crude product. Flash chromatography of the crude product using a prepacked 25 g silica column [eluents; solvent A, EtOAc, solvent B, hexanes; gradient, 15% A/85% B over 1.39 min (1 CV), 15% A/85% B→50% A/50% B over 16.3 min (10 CV), 50% A/50% B over 5.18 min (2 CV); flow rate 25.0 mL/min; monitored at λλ254 and 280 nm] afforded benzosuberene 35 (0.41 g, 0.75 mmol, 78% yield) as a white solid.

¹H NMR (CDCl₃, 500 MHz): δ 7.99 (2H, d, J=8.3 Hz, H-2′, -6′), 7.37 (2H, d, J=8.3 Hz, H-3′, -5′), 6.54 (1H, s, H-4), 6.48 (2H, s, H-2′, H-6′), 6.44 (1H, t, J=7.4 Hz, H-6), 3.87 (3H, s, OCH₃-4′), 3.82 (6H, s, OCH₃-3′, -5′), 3.69 (3H, s, OCH₃-3), 3.54 (3H, s, OCH₃-2), 2.71 (2H, s, CH₂-9), 2.48 (3H, s, CH₃-4″), 2.21 (2H, s, CH₂-8), 1.99 (2H, m, CH₂-7). ¹³C NMR (CDCl₃, 125 MHz): δ 153.0 (C, C-3′, C-5′), 151.0 (C, C-3), 144.7 (C, C-4″), 141.9 (C, C-5), 141.6 (C, C-1), 140.8 (C, C-2), 137.6 (C, C-1′), 137.5 (CH, C-4′), 136.1 (CH, C-4-a), 134.8 (C, C-1″), 129.6 (C, C-1a), 129.5 (C, C-3″, C-5″), 129.4 (CH, C-6), 128.2 (C, C-2″, C-6″), 112.0 (C, C-4), 105.2 (CH, C-2′, C-6′), 60.9 (CH₃, OCH₃-4′), 60.5 (CH₃, OCH₃-2), 56.2 (CH₃, OCH₃-3′, -5′), 56.2 (CH₃, OCH₃-3), 34.6 (CH₂, CH₂-8), 25.5 (CH₂, CH₂-7), 25.1 (CH₂, CH₂-9), 21.7 (CH₃, CH₃-4″).

### 36. 1-Hydroxy-2,3-dimethoxy-5-(3′,4′,5′-trimethoxyphenyl)-6,7,8,9-tetrahydro-5H-cyclobenzoheptan-5-ene (36)

A solution of sulfonate ester 35 (0.250 g, 0.462 mmol) and NaOH (1 mL, 2M) and methanol (4 mL) in a 5 mL microwave safe sealed vial was heated to 100° C. for 1 h. On completion, the reaction mixture was neutralized with 1 mL HCl (2M) and then aqueous solvents were evaporated in vacuo. The crude product was subjected to flash chromatography using a pre-packed silica column affording phenol 36 (0.15 g, 0.388 mmol, 84%) as an off-white solid.

¹H NMR (CDCl₃, 500 MHz): δ 6.51 (2H, s, H-2′, H-6′), 6.39 (1H, t, J=7.4 Hz, H-6), 6.19 (1H, s, H-4), 5.95 (OH, s, OH-1), 3.95 (3H, s, OCH₃-2), 3.87 (3H, s, OCH₃-4′), 3.81 (6H, s, OCH₃-3′, -5′), 3.71 (3H, s, OCH₃-3), 2.68 (2H, s, CH₂-9), 2.13 (2H, s, CH₂-8), 1.96 (2H, m, CH₂-7). ¹³C NMR (CDCl₃, 125 MHz): δ 152.9 (C, C-3′, C-5′), 149.7 (C, C-3), 146.2 (C, C-1), 142.7 (C, C-5), 137.9 (C, C-1′), 136.1 (CH, C-4-a), 137.3 (CH, C-4′), 134.2 (C, C-2), 128.3 (CH, C-6), 121.3 (C, C-1a), 105.2 (CH, C-2′, C-6′), 104.9 (C, C-4), 61.0 (CH₃, OCH₃-2), 60.9 (CH₃, OCH₃-4′), 56.2 (CH₃, OCH₃-3′, -5′), 55.9 (CH₃, OCH₃-2), 34.6 (CH₂, CH₂-8), 25.5 (CH₂, CH₂-7), 25.1 (CH₂, CH₂-9), 21.7 (CH₃, CH₃-4″).

### 37. (E)/(Z) 5-(2′-Fluoro-3′-methoxyphenyl)penta-4-en-1-oic acid (37)

K-OtBu (8.96 g, 79.9 mmol) was added to a well-stirred solution of (3-carboxypropyl)triphenylphosphonium bromide (17.2 g, 40.1 mmol) in THF (250 mL, anhyd) at rt. The reaction mixture was then cooled to 0° C. and stirred for 15 mins. Solution of 2-Fluoro-3-methoxybenzaldehyde (3.08 g, 20.0 mmol) in THF (25 mL, anhyd) was added dropwise to the reaction mixture and the reaction was stirred until the temperature gradually warmed to rt. The reaction was quenched by careful addition of H₂O (50 mL) and extracted with Et₂O (2×250 mL). The aqueous phase was acidified with 2M HCl until the product precipitates making the solution cloudy and then becomes clear again. This acidified aqueous phase was extracted with EtOAc (3×100 mL). The combined organic phases were washed with brine, dried over Na₂SO₄, filtered and evaporated on a rotavapor. Flash chromatography of the crude using a prepacked 50 g silica column, Eluents; solvent A, EtOAc, solvent B, hexanes; gradient, 20% A/80% B over 1.39 min (1 CV), 20% A/80% B→80% A/20% B over 16.3 min (10 CV), 80% A/20% B over 3.18 min (2 CV); flow rate 40.0 mL/min; monitored at A's 254 and 280 nm afforded the mixture of E/Z-isomers 37 (4.14 g, 18.5 mmol, 92% yield), as a colorless liquid.

Anal., Calcd for C₁₂H₁₃FO₃: C, 64.28; H, 5.84. Found: C, 64.49; H, 5.84.

### 38. 5-(2′-Fluoro-3′-methoxyphenyl)pentan-1-oic acid (38)

Through a suspension of 10% Pd/C (400 mg) and pentanoic acid 37 (1.25 g, 20.19 mmol) in EtOH (15 mL), H₂ gas (in balloons) was passed for 12 h. The reaction was checked for completion by filtering a little amount of the reaction mixture through CELITE (diatomaceous earth) and evaporating the solvent to record NMR data. On completion, the reaction mixture was filtered through CELITE (diatomaceous earth) and the solvent was evaporated under reduced pressure to obtain 38 (0.94 g, 2.3 mmol, 75% yield), as an off-white solid.

¹H NMR (500 MHz, CDCl₃) δ 6.99 (1H, t, J=8.2 Hz, H-4′), 6.82 (1H, t, J=8.2 Hz, H-3′), 6.76 (1H, t, J=8.6 Hz, H-5′), 3.88 (3H, s, OCH₃-2′), 2.68 (2H, t, H-5), 2.39 (2H, t, H-2), 1.69 (4H, m, H-3,-4). ¹³C NMR (126 MHz, CDCl₃) δ 179.8 (C, C-1), 150.8 (C, C-1′), 147.7 (C, C-2′), 110.9 (C, C-3′), 129.7 (C, C-6′), 123.6 (CH, C-4′), 129.9 (CH, C-5′), 56.2 (CH₃, OCH₃-2′), 33.7 (CH₂, C-2), 29.5 (CH₂, C-3/4), 28.5 (CH₂, C-5), 24.2 (CH₂, C-4/3). Anal., Calcd for C₁₂H₁₅FO₃: C, 63.70; H, 6.68. Found: C, 63.77; H, 6.70.

### 39. 1-Fluoro-2-methoxy-6,7,8,9-tetrahydro-5H-cyclobenzoheptan-5-one (39)

To pentanoic acid 38 (0.90 g, 2.2 mmol) in a flask, 14 mL of Eatons reagent (from Acros) was added and the solution was stirred for 12 h. The reaction mixture was poured over ice and the ice was allowed to melt. The aqueous phase was extracted with CH₂Cl₂ (3×100 mL) and NaHCO₃ powder was added in small amounts until the pH was neutral. The organic phase was treated with brine, dried over Na₂SO₄, filtered and solvent evaporated under reduced pressure to obtain 39 as white solid. Flash chromatography of the crude using a prepacked 25 g silica column, Eluents; solvent A, EtOAc, solvent B, hexanes; gradient, 25% A/75% B over 3.18 min (1 CV), 25% A/75% B→60% A/40% B over 33.0 min (10 CV), 60% A/40% B over 6.36 min (2 CV); flow rate 25.0 mL/min; monitored at λλ254 and 280 nm afforded 39, (0.70 g, 1.8 mmol, 81% yield) as a white solid.

¹H NMR (500 MHz, CDCl₃) δ 7.57 (1H, d, J=8.7, 1.7 Hz, H-4′), 6.88 (1H, t, J=8.3, Hz, H-3′), 3.93 (3H, s, OCH₃-2), 3.00 (2H, m, H-9), 2.72 (2H, m, CH₃-6), 1.87 (2H, m, H-8), 1.82 (2H, m, H-7). ¹³C NMR (126 MHz, CDCl₃) δ 203.6 (C, C-5), 110.2 (C, C-3), 151.0 (C, C-2), 125.0 (C, C-4), 149.2 (C, C-1), 132.4 (C, C-4-a), 129.6 (C, C-1a), 56.2 (CH₃, OCH₃-2), 40.6 (CH₂, C-6), 24.8 (CH₂, C-8), 22.5 (CH₂, C-9), 20.9 (CH₂, C-7). Anal., Calcd for C₁₂H₁₃FO₂: C, 69.22; H, 6.29. Found: C, 69.00; H, 6.30.

### 40. 1-Fluoro-2-methoxy-5-(3′,4′,5′-trimethoxyphenyl)-6,7,8,9-tetrahydro-5H-cyclobenzoheptan-5-ol (40)

To a solution of 3,4,5-trimethoxyphenylbromide (0.85 g, 3.4 mmol) in anhydrous THF (100 mL) at −78° C., n-BuLi (1.4 mL, 2.5 M in hexanes) was added and the reaction stirred for 30 min. Benzosuberone 39 (0.67 g, 1.7 mmol) in 15 mL THF was added using a dropping funnel over a period of 15 min. The reaction mixture was stirred overnight and allowed to warm to ambient temperature. On completion, the reaction mixture was quenched with H₂O (100 mL), and extracted with Et₂O (150 mL) and EtOAc (15 mL). The combined organic phase was washed with brine, dried over MgSO₄, filtered, and evaporated under reduced pressure. Flash chromatography of the crude product using a prepacked 25 g silica column [eluents; solvent A, EtOAc, solvent B, hexanes; gradient, 25% A/75% B over 1.39 min (1 CV), 25% A/75% B 80% A/20% B over 16.3 min (10 CV), 80% A/20% B over 5.18 min (2 CV); flow rate 25.0 mL/min; monitored at λλ254 and 280 nm] afforded alcohol 40 (0.61 g, 1.1 mmol, 64% yield) as a white solid.

¹H NMR (CDCl₃, 500 MHz): δ 7.27 (1H, d, J=8.8 Hz, H-4), 6.79 (1H, d, J=8.7 Hz, H-3), 6.48 (2H, s, H-2′, H-6′), 3.90 (3H, s, OCH₃-2), 3.85 (3H, s, OCH₃-4′), 3.76 (6H, s, OCH₃-3′, -5′), 3.16 (1H, m, CH₂-9), 2.37 (1H, m, CH₂-9), 2.57 (1H, m, CH₂-8). 2.11 (1H, m, CH₂-8), 1.94 (1H, s, CH₂-716), 1.77 (2H, m, CH₂-6, CH₂-7), 1.49 (1H, m, H-6/7). ¹³C NMR (CDCl₃, 125 MHz): δ 153.1 (C, C-3′, C-5′), 149.6 (C, J_{C-F}=2.0 ppm, C-1), 146.7 (C, C-2), 141.1 (C, C-1′), 137.4 (CH, C-4′), 138.8 (CH, C-4-a), 129.1 (C, C-1a), 122.1 (C, C-4), 109.4 (C, C-1), 104.2 (CH, C-2′, C-6′), 79.8 (C, C-5), 60.8 (CH₃, OCH₃-4′), 56.1 (CH₃, OCH₃-3′, -5′), 56.0 (CH₃, OCH₃-2), 41.2 (CH, C-6), 26.6 (CH₂, CH₂-817), 26.2 (CH₂, CH₂-718), 24.2 (CH₂, CH₂-9). ¹⁹F NMR (CDCl₃, 125 MHz): δ 142.9. Anal., Calcd for C₂₁H₂₅FO₅: C, 67.01; H, 6.69. Found: C, 67.11; H, 6.66.

### 41. 1-Fluoro-2-methoxy-5-(3′,4′,5′-trimethoxyphenyl)-6,7,8,9-tetrahydro-5H-cyclobenzoheptan-5-ene (41)

A solution of alcohol 40 (1.27 g, 0.97 mmol) in AcOH (20 mL) and H₂O (30 mL) was heated to reflux at 150° C. for 12 h. The reaction mixture was cooled and the aqueous solvents evaporated over a reduced pressure to obtain a crude product. Flash chromatography of the crude product using a prepacked 25 g silica column [eluents; solvent A, EtOAc, solvent B, hexanes; gradient, 10% A/90% B over 1.39 min (1 CV), 10% A/90% B→50% A/50% B over 16.3 min (10 CV), 50% A/50% B over 5.18 min (2 CV); flow rate 25.0 mL/min; monitored at λλ254 and 280 nm] afforded benzosuberene 41 (1.07 g, 0.75 mmol, 78% yield) as a white solid.

¹H NMR (CDCl₃, 500 MHz): δ 6.80-6.78 (2H, m, H-3, H-4), 6.48 (2H, s, H-2′, H-6′), 6.36 (1H, t, J=7.4 Hz, H-6), 3.91 (3H, s, OCH₃-2), 3.86 (3H, s, OCH₃-4′), 3.81 (6H, s, OCH₃-3′, -5′), 2.74 (2H, s, CH₂-9), 2.16 (2H, s, CH₂-8), 1.97 (2H, m, CH₂-7). ¹³C NMR (CDCl₃, 125 MHz): δ 152.9 (C, C-3′, C-5′), 149.6 (C, J_{C-F}=2.0 ppm, C-1), 146.4 (C, C-2), 142.1 (C, C-5), 138.0 (C, C-1′), 137.4 (CH, C-4′), 133.9 (CH, C-4-a), 129.6 (C, C-1a), 127.6 (CH, C-6), 124.8 (C, C-4), 110.0 (C, C-1), 105.2 (CH, C-2′, C-6′), 60.9 (CH₃, OCH₃-4′), 56.1 (CH₃, OCH₃-3′, -5′), 56.1 (CH₃, OCH₃-2), 33.6 (CH₂, CH₂-8), 25.5 (CH₂, CH₂-7), 23.2 (CH₂, CH₂-9). ¹⁹F NMR (CDCl₃, 125 MHz): δ 142.9.

### 42. (E)/(Z) 5-(2′-Chloro-3′-methoxyphenyl)penta-4-en-1-oic acid (42)

K-OtBu (6.99 g, 62.3 mmol) was added to a well-stirred solution of (3-carboxypropyl)triphenylphosphonium bromide (13.1 g, 30.5 mmol) in THF (250 mL, anhyd) at rt. The reaction mixture was then cooled to 0° C. and stirred for 15 mins. Solution of 2-Chloro-3-methoxybenzaldehyde (3.44 g, 20.2 mmol) in THF (25 mL, anhyd) was added dropwise to the reaction mixture and the reaction was stirred until the temperature gradually warmed to r.t. The reaction was quenched by careful addition of H₂O (50 mL) and extracted with Et₂O (2×250 mL). The aqueous phase was acidified with 2M HCl until the product precipitates making the solution cloudy and then becomes clear again. This acidified aqueous phase was extracted with EtOAc (3×100 mL). The combined organic phases were washed with brine, dried over Na₂SO₄, filtered and evaporated on a rotavapor. Flash chromatography of the crude using a prepacked 50 g silica column, Eluents; solvent A, EtOAc, solvent B, hexanes; gradient, 40% A/60% B over 1.39 min (1 CV), 40% A/60% B→80% A/20% B over 16.3 min (10 CV), 60% A/40% B over 3.18 min (2 CV); flow rate 40.0 mL/min; monitored at λλ254 and 280 nm afforded the mixture of E/Z-isomers 42 (4.80 g, 19.9 mmol, 98% yield), as a colorless liquid.

### 43. 5-(2′-Chloro-3′-methoxyphenyl)pentan-1-oic acid (43)

Through a suspension of 10% Pd/C (0.729 g) and 5-(1′-chloro-2′-methoxyphenyl) penta-4-en-1-oic acid (4.87 g, 20.2 mmol) in EtOH (50 mL), H₂ gas (in balloons) was passed for 12 h. The reaction was monitored for completion by filtering a little amount of the reaction mixture through CELITE (diatomaceous earth) and evaporating the solvent to record NMR data. On completion, the reaction mixture was filtered through CELITE (diatomaceous earth) and the solvent was evaporated under reduced pressure to obtain 43 (2.55 g, 2.3 mmol, 75% yield), as an off-white solid.

¹H NMR (500 MHz, CDCl₃) δ 7.14 (1H, t, J=8.2, 7.7 Hz, H-4′), 6.84 (1H, t, J=7.7 Hz, H-5′), 6.79 (1H, t, J=8.2 Hz, H-3′), 3.89 (3H, s, OCH₃-2′), 2.77 (2H, t, J=6.9, 7.5 Hz, H-5), 2.40 (2H, t, J=6.5, 7.2 Hz, H-2), 1.70 (4H, m, H-3,-4). ¹³C NMR (126 MHz, CDCl₃) δ 179.3 (C, C-1), 155.2 (C, C-2′), 141.3 (C, C-6′), 109.6 (C, C-3′), 122.2 (C, C-1′), 126.8 (CH, C-4′), 122.2 (CH, C-5′), 56.2 (CH₃, OCH₃-2′), 33.8 (CH₂, C-2), 33.3 (CH₂, C-5), 29.0 (CH₂, C-3/4), 24.4 (CH₂, C-4/3). Anal., Calcd for C₁₂H₁₅ClO₃: C, 59.39; H, 6.23. Found: C, 59.57; H, 6.28.

### 44. 1-Chloro-2-methoxy-6,7,8,9-tetrahydro-5H-cyclobenzoheptan-5-one (44)

To pentanoic acid 43 (2.50 g, 10.3 mmol) in a flask, 55 mL of Eatons reagent (from Acros) was added and the solution was stirred for 12 h. The reaction mixture was poured over ice and the ice was allowed to melt. The aqueous phase was extracted with CH₂Cl₂ (3×150 mL) and NaHCO₃ powder was added in small amounts until the pH was neutral. The organic phase was treated with brine, dried over Na₂SO₄, filtered and solvent evaporated under reduced pressure to obtain 44 as white solid. Flash chromatography of the crude using a prepacked 50 g silica column, Eluents; solvent A, EtOAc, solvent B, hexanes; gradient, 10% A/90% B over 3.18 min (1 CV), 10% A/90% B→60% A/40% B over 33.0 min (10 CV), 60% A/40% B over 6.36 min (2 CV); flow rate 40.0 mL/min; monitored at λλ254 and 280 nm afforded 44, (2.23 g, 9.93 mmol, 96% yield) as a white solid.

¹H NMR (CDCl₃, 500 MHz): δ 7.61 (1H, d, J=8.7 Hz, H-4′), 6.86 (1H, d, J=8.7, Hz, H-3′), 3.95 (3H, s, OCH₃-2), 3.14 (2H, m, H-9), 2.69 (2H, m, CH₃-6), 1.86 (2H, m, H-8), 1.78 (2H, m, H-7). ¹³C NMR (CDCl₃, 500 MHz): δ 204.7 (C, C-5), 157.9 (C, C-2), 140.3 (C, C-1), 133.3 (C, C-4-a), 128.1 (C, C-1a), 122.0 (C, C-4), 109.3 (C, C-3), 56.4 (CH₃, OCH₃-2), 40.5 (CH₂, C-6), 27.8 (CH₂, C-8), 23.8 (CH₂, C-9), 20.7 (CH₂, C-7). Anal., Calcd for C₁₂H₁₃ClO₃: C, 64.15; H, 5.83. Found: C, 64.25; H, 5.85.

### 45. 1-Chloro-2-methoxy-5-(3′,4′,5′-trimethoxyphenyl)-6,7,8,9-tetrahydro-5H-cyclobenzoheptan-5-ol (45)

To a solution of 3,4,5-trimethoxyphenylbromide (3.60 g, 14.6 mmol) in anhydrous THF (250 mL) at −78° C., n-BuLi (6.0 mL, 2.5 M in hexanes) was added and the reaction stirred for 30 min. Benzosuberone 44 (1.87 g, 8.32 mmol) in 15 mL THF was added using a dropping funnel over a period of 15 min. The reaction mixture was stirred overnight and allowed to warm to ambient temperature. On completion, the reaction mixture was quenched with H₂O (100 mL), and extracted with EtOAc (2×200 mL). The combined organic phase was washed with brine, dried over MgSO₄, filtered, and evaporated under reduced pressure. Flash chromatography of the crude product using a prepacked 50 g silica column [eluents; solvent A, EtOAc, solvent B, hexanes; gradient, 10% A/90% B over 1.39 min (1 CV), 10% A/90% B→60% A/40% B over 16.3 min (10 CV), 60% A/40% B over 5.18 min (2 CV); flow rate 40.0 mL/min; monitored at λλ254 and 280 nm] afforded alcohol 45 (2.41 g, 6.13 mmol, 74% yield) as an off-white solid.

¹H NMR (CDCl₃, 500 MHz): δ 7.50 (1H, d, J=8.8 Hz, H-4), 6.80 (1H, d, J=8.8 Hz, H-3), 6.49 (2H, s, H-2′, H-6′), 3.92 (3H, s, OCH₃-2), 3.84 (3H, s, OCH₃-4′), 3.76 (6H, s, OCH₃-3′, -5′), 3.41 (1H, m, CH₂-9), 2.51 (1H, m, CH₂-9), 2.58 (1H, m, CH₂-6), 2.12 (1H, m, CH₂-6), 1.88 (2H, s, CH₂-7), 1.76 (2H, m, CH₂-6, CH₂-8), 1.47 (1H, m, H-8). ¹³C NMR (CDCl₃, 125 MHz): δ 154.1 (C, C-2), 153.1 (C, C-3′, C-5′), 141.3 (C, C-1′), 140.1 (C, C-1a), 138.9 (CH, C-4-a), 137.5 (CH, C-4′), 125.9 (C, C-4), 122.4 (C, C-1), 108.6 (C, C-3), 104.1 (CH, C-2′, C-6′), 79.9 (C, C-5), 60.8 (CH₃, OCH₃-4′), 56.2 (CH₃, OCH₃-3′, -5′), 56.1 (CH₃, OCH₃-2), 41.1 (CH, C-6), 29.5 (CH₂, CH₂-9), 25.9 (CH₂, CH₂-7), 25.8 (CH₂, CH₂-8). Anal., Calcd for C₂₁H₂₅ClO₅: C, 64.20; H, 6.41. Found: C, 64.41; H, 6.45.

### 46. 1-Chloro-2-methoxy-5-(3′,4′,5′-trimethoxyphenyl)-6,7,8,9-tetrahydro-5H-cyclobenzoheptan-5-ene (46)

A solution of alcohol 45 (2.37 g, 6.03 mmol) in AcOH (50 mL) and H₂O (50 mL) was heated to reflux at 110° C. for 12 h. The reaction mixture was cooled and the aqueous solvents evaporated over a reduced pressure to obtain a crude product. Flash chromatography of the crude product using a prepacked 25 g silica column [eluents; solvent A, EtOAc, solvent B, hexanes; gradient, 10% A/90% B over 1.39 min (1 CV), 10% A/90% B→60% A/40% B over 16.3 min (10 CV), 60% A/40% B over 5.18 min (2 CV); flow rate 50.0 mL/min; monitored at λλ254 and 280 nm] afforded benzosuberene 46 (2.18 g, 5.81 mmol, 97% yield) as a white solid.

¹H NMR (CDCl₃, 500 MHz): δ 6.94 (1H, d, H-3, H-4), 6.80 (1H, d, H-3, H-4), 6.49 (2H, s, H-2′, H-6′), 6.39 (1H, t, J=7.4 Hz, H-6), 3.93 (3H, s, OCH₃-2), 3.87 (3H, s, OCH₃-4′), 3.82 (6H, s, OCH₃-3′, -5′), 2.91 (2H, t, J=7.0 Hz, CH₂-9), 2.19 (2H, p, J=7.2, 7.0 Hz, CH₂-8), 1.94 (2H, t, J=7.3, 7.2 Hz, CH₂-7). ¹³C NMR (CDCl₃, 125 MHz): δ 153.9 (C, C-2), 152.9 (C, C-3′, C-5′), 142.6 (C, C-5), 141.0 (C, C-1a), 137.9 (C, C-1′), 137.5 (CH, C-4′), 134.1 (CH, C-4-a), 127.6 (CH, C-6), 128.1 (C, C-4), 121.4 (C, C-1), 109.0 (C, C-3), 105.1 (CH, C-2′, C-6′), 60.9 (CH₃, OCH₃-4′), 56.18 (CH₃, OCH₃-2), 56.15 (CH₃, OCH₃-3′, -5′), 33.7 (CH₂, CH₂-8), 25.4 (CH₂, CH₂-7), 28.6 (CH₂, CH₂-9). Anal., Calcd for C₂₁H₂₃ClO₄: C, 67.29; H, 6.18. Found: C, 67.20; H, 6.18.

### 47. 5-(3′-Methoxyphenyl)pentan-1-oic acid (47)

Through a suspension of 10% Pd/C (0.329 g) and 5-(3′-methoxyphenyl)penta-4-en-1-oic acid 56 (3.13 g, 15.2 mmol) in EtOH (50 mL), H₂ gas (in balloons) was passed for 12 h. The reaction was checked for completion by filtering a little amount of the reaction mixture through CELITE (diatomaceous earth) and evaporating the solvent to record NMR data. On completion, the reaction mixture was filtered through CELITE (diatomaceous earth) and the solvent was evaporated under reduced pressure to obtain 47 (3.11 g, 14.9 mmol, 98% yield), as an off-white solid.

¹H NMR (500 MHz, CDCl₃) δ 7.14 (1H, t, J=8.2, 7.7 Hz, H-4′), 6.84 (1H, t, J=7.7 Hz, H-5′), 6.79 (1H, t, J=8.2 Hz, H-3′), 3.89 (3H, s, OCH₃-2′), 2.77 (2H, t, J=6.9, 7.5 Hz, H-5), 2.40 (2H, t, J=6.5, 7.2 Hz, H-2), 1.70 (4H, m, H-3,-4). ¹³C NMR (126 MHz, CDCl₃) δ 179.3 (C, C-1), 155.2 (C, C-2′), 141.3 (C, C-6′), 109.6 (C, C-3′), 122.2 (C, C-1′), 126.8 (CH, C-4′), 122.2 (CH, C-5′), 56.2 (CH₃, OCH₃-2′), 33.8 (CH₂, C-2), 33.3 (CH₂, C-5), 29.0 (CH₂, C-3/4), 24.4 (CH₂, C-4/3).

### 48. 2-Methoxy-6,7,8,9-tetrahydro-5H-cyclobenzoheptan-5-one (48)

To pentanoic acid 47 (3.01 g, 14.5 mmol) in a flask, 40 g/mL of Eatons reagent (from Acros) was added and the solution was stirred for 12 h. The reaction mixture was poured over ice and the ice was allowed to melt. The aqueous phase was extracted with CH₂Cl₂ (3×100 mL) and NaHCO₃ powder was added in small amounts until the pH was neutral. The organic phase was treated with brine, dried over Na₂SO₄, filtered and solvent evaporated under reduced pressure to obtain 48 as a white solid. Flash chromatography of the crude using a prepacked 50 g silica column, Eluents; solvent A, EtOAc, solvent B, hexanes; gradient, 10% A/90% B over 3.18 min (1 CV), 10% A/90% B→60% A/40% B over 33.0 min (10 CV), 60% A/40% B over 6.36 min (2 CV); flow rate 40.0 mL/min; monitored at λλ254 and 280 nm afforded 48, (2.10 g, 11.0 mmol, 76% yield) as a white solid.

¹H NMR (CDCl₃, 500 MHz): δ 7.78 (1H, d, J=8.6 Hz, H-4′), 6.81 (1H, dd, J=8.6, 2.5 Hz, H-3′), 6.70 (1H, d, J=2.5 Hz, H-4′), 3.85 (3H, s, OCH₃-2), 2.91 (2H, m, H-9), 2.71 (2H, m, CH₃-6), 1.89 (2H, m, H-8), 1.81 (2H, m, H-7). ¹³C NMR (CDCl₃, 500 MHz): δ 204.3 (C, C-5), 162.7 (C, C-2), 144.2 (C, C-1a), 131.6 (C, C-4-a), 131.3 (C, C-4), 114.9 (C, C-1), 111.7 (C, C-3), 55.3 (CH₃, OCH₃-2), 40.7 (CH₂, C-6), 32.9 (CH₂, C-9), 25.1 (CH₂, C-8), 20.7 (CH₂, C-7).

### 49. 2-Methoxy-5-(3′,4′,5′-trimethoxyphenyl)-6,7,8,9-tetrahydro-5H-cyclobenzoheptan-5-ol (49)

To a solution of 3,4,5-trimethoxyphenylbromide (5.05 g, 20.4 mmol) in anhydrous THF (150 mL) at −78° C., n-BuLi (8.5 mL, 2.5 M in hexanes) was added and the reaction stirred for 30 min. Benzosuberone 48 (2.10 g, 11.0 mmol) in 25 mL THF was added using a dropping funnel over a period of 15 min. The reaction mixture was stirred overnight and allowed to warm to ambient temperature. On completion, the reaction mixture was quenched with H₂O (100 mL), and extracted with Et₂O (150 mL) and EtOAc (15 mL). The combined organic phase was washed with brine, dried over MgSO₄, filtered, and evaporated under reduced pressure. Flash chromatography of the crude product using a prepacked 25 g silica column [eluents; solvent A, EtOAc, solvent B, hexanes; gradient, 25% A/75% B over 1.39 min (1 CV), 25% A/75% B 80% A/20% B over 16.3 min (10 CV), 80% A/20% B over 5.18 min (2 CV); flow rate 25.0 mL/min; monitored at λλ254 and 280 nm] afforded alcohol 49 (3.81 g, 10.6 mmol, 96% yield) as a white solid.

¹H NMR (CDCl₃, 500 MHz): δ 7.27 (1H, d, J=8.8 Hz, H-4), 6.79 (1H, d, J=8.7 Hz, H-3), 6.48 (2H, s, H-2′, H-6′), 3.90 (3H, s, OCH₃-2), 3.85 (3H, s, OCH₃-4′), 3.76 (6H, s, OCH₃-3′, -5′), 3.16 (1H, m, CH₂-9), 2.37 (1H, m, CH₂-9), 2.57 (1H, m, CH₂-8). 2.11 (1H, m, CH₂-8), 1.94 (1H, s, CH₂-716), 1.77 (2H, m, CH₂-6, CH₂-7), 1.49 (1H, m, H-6/7). ¹³C NMR (CDCl₃, 125 MHz): δ 153.1 (C, C-3′, C-5′), 149.6 (C, J_{C-F}=2.0 ppm, C-1), 146.7 (C, C-2), 141.1 (C, C-1′), 137.4 (CH, C-4′), 138.8 (CH, C-4-a), 129.1 (C, C-1a), 122.1 (C, C-4), 109.4 (C, C-1), 104.2 (CH, C-2′, C-6′), 79.8 (C, C-5), 60.8 (CH₃, OCH₃-4′), 56.1 (CH₃, OCH₃-3′, -5′), 56.0 (CH₃, OCH₃-2), 41.2 (CH, C-6), 26.6 (CH₂, CH₂-817), 26.2 (CH₂, CH₂-718), 24.2 (CH₂, CH₂-9).

### 50. 2-Methoxy-5-(3′,4′,5′-trimethoxyphenyl)-6,7,8,9-tetrahydro-5H-cyclobenzoheptan-5-ene (50)

A solution of alcohol 49 (3.80 g, 10.6 mmol) in AcOH (25 mL) and H₂O (25 mL) was heated to reflux at 160° C. for 12 h. The reaction mixture was cooled and the aqueous solvents evaporated over a reduced pressure to obtain a crude product. Flash chromatography of the crude product using a prepacked 25 g silica column [eluents; solvent A, EtOAc, solvent B, hexanes; gradient, 10% A/90% B over 1.39 min (1 CV), 10% A/90% B→50% A/50% B over 16.3 min (10 CV), 50% A/50% B over 5.18 min (2 CV); flow rate 25.0 mL/min; monitored at λλ254 and 280 nm] afforded benzosuberene 50 (1.07 g, 9.17 mmol, 86% yield) as a white solid.

¹H NMR (CDCl₃, 500 MHz): δ 6.98 (1H, d, J=7.4 Hz, H-4), 6.83 (1H, d, H-1), 6.74 (1H, dd, J=7.4 Hz, H-6), 6.49 (2H, s, H-2′, H-6′), 6.35 (1H, t, H-6), 3.86 (3H, s, OCH₃-2), 3.84 (3H, s, OCH₃-4′), 3.80 (6H, s, OCH₃-3′, -5′), 2.64 (2H, s, CH₂-9), 2.17 (2H, s, CH₂-8), 1.97 (2H, m, CH₂-7). ¹³C NMR (CDCl₃, 125 MHz): δ 152.9 (C, C-3′, C-5′), 149.6 (C, J_{C-F}=2.0 ppm, C-1), 146.4 (C, C-2), 142.1 (C, C-5), 138.0 (C, C-1′), 137.4 (CH, C-4′), 133.9 (CH, C-4-a), 129.6 (C, C-1a), 127.6 (CH, C-6), 124.8 (C, C-4), 110.0 (C, C-1), 105.2 (CH, C-2′, C-6′), 60.9 (CH₃, OCH₃-4′), 56.1 (CH₃, OCH₃-3′, -5′), 56.1 (CH₃, OCH₃-2), 33.6 (CH₂, CH₂-8), 25.5 (CH₂, CH₂-7), 23.2 (CH₂, CH₂-9). Anal., Calcd for C₂₁H₂₄O₄: C, 74.09; H, 7.11. Found: C, 74.06; H, 7.08.

### 51. 2-Hydroxy-6,7,8,9-tetrahydro-5H-cyclobenzoheptan-5-one (51)

2-Methoxy-6,7,8,9-tetrahydro-5H-cyclobenzoheptan-5-one (48) (0.505 g, 2.66 mmol) was added to 5 mL microwave vial with stir bar. To the vial was added [TMAH][Al₂Cl₇] solution (5.65 mL, 0.9423 M, 5.32 mmol). The vial was sealed and placed in a microwave reaction chamber for 1 h at 80° C. with 30 sec of pre-stirring. The reaction mixture was then slowly added to H₂O (20 mL). The aqueous reaction mixture was extracted with EtOAc (4×25 mL). The combined organic layers were washed with brine, dried with Na₂SO₄, evaporated under reduced pressure and purified by flash chromatography with a prepacked 50 g silica gel column [eluents; solvent A, EtOAc, solvent B, hexanes; gradient; 5% A/95% B→7% A/93% B (1 CV), 7% A/93% B→60% A/40% B (12 CV), 60% A/40% B (5.5 CV); flow rate 40 mL/min; monitored at λλ254 and 280 nm]. Alcohol analog 51 (0.407 g, 2.31 mmol, 87% yield) was obtained as a light pink solid, R_{f}=0.24 (70:30 Hexanes:EtOAc).

¹H NMR (DMSO, 500 MHz): δ 10.10 (1H, s, H=Ar—OH), 7.54 (1H, d, J=8.6 Hz, H=Ar—H), 6.68 (1H, dd, J=8.6, 2.2 Hz, H=Ar—H), 6.63 (1H, d, J=2.2 Hz, H=Ar—H), 2.83 (2H, t, J=6.2 Hz, H═CH₂), 2.61 (2H, t, J=6.0 Hz, H═CH₂), 1.75 (2H, p, J=6.5 Hz, H═CH₂), 1.66 (2H, p, J=6.2 Hz, H═CH₂). ¹³C NMR (DMSO, 125 MHz): δ 203.0, 161.6, 145.0, 131.3, 130.0, 116.6, 113.9, 40.6, 32.3, 25.0, 20.6.

### 52. 2-((tert-Butyldimethylsilyl)oxy)-6,7,8,9-tetrahydro-5H-cyclobenzoheptan-5-one (52)

7-hydroxybenzosuberone analog 51 (0.407 g, 2.31 mmol) was dissolved in DMF (10 mL) in a flask with stir bar. To the flask was added TBS-CI (0.513 g, 3.40 mmol) and DIPEA (0.82 mL, 4.71 mmol). The solution was stirred for 12 h at ambient temperature. The reaction was quenched with H₂O (20 mL) then extracted with EtOAc (3×25 mL). The combined organic layers were washed with H₂O (2×30 ml), brine, dried with Na₂SO₄, evaporated under reduced pressure and purified by flash chromatography with a prepacked 50 g silica gel column [eluents; solvent A, EtOAc, solvent B, hexanes; gradient; 0% A/100% B→2% A/98% B (1 CV), 2% A/98% B→16% A/84% B (9 CV); flow rate 40 mL/min; monitored at λλ254 and 280 nm]. TBS-protected analog 52 (0.546 g, 1.88 mmol, 81% yield) was obtained as a white solid, R_{f}=0.39 (90:10 Hexanes:EtOAc).

¹H NMR (CDCl₃, 500 MHz): δ 7.71 (1H, d, J=7.6 Hz, H=Ar—H), 6.74 (1H, dd, J=8.6, 2.4 Hz, H=Ar—H), 6.65 (1H, d, J=2.2 Hz, H=Ar—H), 2.87 (2H, m, H═CH₂), 2.70 (2H, m, H═CH₂), 1.86 (2H, m, H═CH₂), 1.79 (2H, m, H═CH₂). ¹³C NMR (CDCl₃, 125 MHz): δ 204.4, 159.2, 144.1, 132.1, 130.9, 120.9, 118.0, 40.7, 32.6, 25.5, 25.0, 20.7, 18.2, −4.4.

### 53. 2-((tert-Butyldimethylsilyl)oxy)-5-(3′,4′,5′-trimethoxyphenyl)-6,7,8,9-tetrahydro-5H-cyclobenzoheptan-5-ol (53)

A solution of 3,4,5-trimethoxyphenylbromide (0.595 g, 2.41 mmol) in anhydrous THF (25 mL) under N₂ was cooled to −78° C., then n-BuLi (0.97 mL, 2.5 M in hexanes) was added and stirred for 1 h. Ketone 52 (0.546 g, 1.88 mmol) was slowly added to the reaction mixture in anhydrous THF (3 mL) and allowed to warm to ambient temperature overnight with continuous stirring. On completion, the reaction mixture was quenched with H₂O (10 mL), and the solvent was evaporated under reduced pressure. The aqueous phase was extracted using EtOAc (4×20 mL). The combined organic extracts were washed with brine, dried over Na₂SO₄, filtered, evaporated under reduced pressure, and purified by flash chromatography using a prepacked 50 g silica column [eluents; solvent A, EtOAc, solvent B, hexanes; gradient, 5% A/95% B (1 CV), 5% A/95% B→60% A/40% B (13 CV), 60% A/40% B (1.5 CV); flow rate, 35 mL/min; monitored at λλ254 and 280 nm]. Tertiary alcohol 53 (0.708 g, 1.54 mmol, 64% yield) was obtained as a clear oil, R_{f} 0.37 (70:30, hexanes:EtOAc).

¹H NMR (CDCl₃, 500 MHz): δ 7.35 (1H, d, J=8.7 Hz, H=Ar—H), 6.65 (1H, dd, J=8.6, 2.7 Hz, H=Ar—H), 6.60 (1H, d, J=2.7, H=Ar—H), 6.45 (2H, s, H=(Ar—H)₂), 3.81 (3H, s, H═OCH₃), 3.71 (6H, s, H═(OCH₃)₂), 2.67 (1H, m, H═CH), 2.53 (2H, m, H═CH₂), 2.33 (1H, s, H ═OH), 2.09 (1H, m, H═CH), 1.90 (1H, m, H═CH), 1.70 (2H, m, H═CH₂), 1.52 (1H, m, H═CH), 0.972 (9H, s, —Si(CH₃)₃), 0.191 (6H, s, —Si(CH₃)₃). ¹³C NMR (CDCl₃, 125 MHz): δ 154.5, 152.8, 142.5, 141.7, 138.0, 137.0, 128.5, 122.0, 116.9, 104.1, 79.7, 60.7, 55.8, 41.2, 36.2, 27.2, 25.8, 25.6, 18.1, −4.5

### 54. 2-((tert-Butyldimethylsilyl)oxy)-5-(3′,4′,5′-trimethoxyphenyl)-6,7,8,9-tetrahydro-5H-cyclobenzohepta-6-ene (54)

Tertiary alcohol 53 (0.708 g, 1.54 mmol) was dissolved in AcOH (10 mL) and stirred overnight at ambient temperature. The reaction was quenched with H₂O (30 mL). The reaction mixture was then extracted with EtOAc (3×20 mL). The combined organic extracts were washed with sat. NaHCO₃, brine, dried over Na₂SO₄, filtered, evaporated under reduced pressure, and purified by flash chromatography using a prepacked 50 g silica gel column [eluent; solvent A, EtOAc, solvent B, hexanes; gradient 0% A/100% B→2% A/98% B (1 CV), 2% A/98% B→18% A/82% B (10.5 CV); flow rate, 35 mL/min; monitored at λλ254 and 280 nm]. TBS-protected benzylidene analog 54 (0.459 g, 1.04 mmol, 67% yield) was obtained as a clear oil, R_{f}=0.32 (90:10, hexanes:EtOAc).

¹H NMR (CDCl₃, 500 MHz): δ 6.90 (1H, d, J=8.3 Hz), 6.77 (1H, d, J=2.4 Hz), 6.67 (1 H, dd, J=8.3, 2.4, H=Ar—H), 6.48 (2H, s), 6.34 (1H, t, J=7.3 Hz), 3.86 (3H, s), 3.79 (6H, s), 2.60 (2H, t, J=7.0 Hz), 2.15 (2H, p, J=7.1 Hz), 1.96 (2H, q, J=7.2 Hz), 1.00 (9H, s, H=TBS-(CH₃)₃), 0.230 (6H, s, H=TBS-(CH₃)₂). ¹³C NMR (CDCl₃, 125 MHz): δ 154.4, 152.8, 143.7, 142.7, 138.4, 137.3, 133.0, 130.4, 127.0, 120.0, 117.3, 105.2, 60.9, 56.0, 35.0, 32.6, 25.6, 25.4, 18.2, −4.3.

### 55. 2-Hydroxy-5-(3′,4′,5′-trimethoxyphenyl)-6,7,8,9-tetrahydro-5H-cyclobenzohepta-6-ene (55)

TBS-protected analog 54 (0.459 g, 1.04 mmol) was added to a flask containing THF (5 mL). To the solution was added TBAF (1.1 mL, 1M). The solution was stirred for 1 h at room temperature. The reaction was quenched with H₂O (10 mL) and the organic solvent was removed under reduced pressure. The aqueous phase was then extracted with EtOAc (4×10 mL). The combined organic extracts were washed with brine, dried over Na₂SO₄, filtered, evaporated under reduced pressure, and purified by flash chromatography using a prepacked 50 g silica gel column [eluent; solvent A, EtOAc, solvent B, hexanes; gradient 5% A/95% B 7% A/93% B (1 CV), 7% A/93% B→60% A/40% B (12 CV), 60% A/40% B (2 CV); flow rate, 40 mL/min; monitored at λλ254 and 280 nm]. Phenol analog 55 (0.264 g, 0.810 mmol, 78% yield) was obtained as a white solid, R_{f}=0.29 (70:30, hexanes:EtOAc).

¹H NMR (CDCl₃, 500 MHz): δ 6.92 (1H, d, J=8.3 Hz), 6.77 (1H, d, J=2.7 Hz), 6.66 (1H, dd, J=8.6, 2.7), 6.49 (2H, s), 6.34 (1H, t, J=7.8 Hz), 5.11 (1H, s), 3.86 (3H, s), 3.80 (6H, s), 2.61 (2H, t, J=7.0 Hz), 2.15 (2H, p, J=7.1 Hz), 1.97 (2H, q, J=7.2 Hz). ¹³C NMR (CDCl₃, 125 MHz): δ 154.5, 152.8, 144.1, 142.6, 138.5, 137.3, 132.5, 130.8, 127.1, 115.3, 112.8, 105.2, 60.9, 56.1, 35.0, 32.6, 25.5.

### 56. 5-(3′-Methoxyphenyl)pent-4-enoic acid (56)

To a solution of THF (500 mL) was added (3-carboxypropyl)triphenylphosphonium bromide (18.92 g, 44.1 mmol) and potassium tert-butoxide (10.50 g, 93.6 mmol). The solution was allowed to stir for 1 h at ambient temperature. 3-Methoxybenzaldehyde (5.38 g, 39.5 mmol) was dissolved into THF (10 mL) then added drop-wise to the reaction mixture in anhydrous THF (5 mL), which was allowed to stir for 12 h at ambient temperature. The solution was quenched with 2M HCl (25 mL), and the organic solvent was evaporated under reduced pressure. The aqueous phase was extracted with EtOAc (4×50 mL), washed with brine, dried over Na₂SO₄, filtered, evaporated under reduced pressure, and purified by flash chromatography with a prepacked 100 g silica gel column [eluents; solvent A, EtOAc, solvent B, hexanes; gradient, 10% A/90% B→12% A/88% B (1 CV), 12% A/88% B→71% A/29% B (8 CV); flow rate, 40 mL/min; monitored at λλ254 and 280 nm]. Pentenoic acid analog 56 (0.879 g, 3.46 mmol, 77% yield) was obtained as a mixture of E and Z isomers and was a yellow solid.

¹H NMR (CDCl₃, 500 MHz) Reported as E & Z mixture: δ 7.26 (1H, t, J=7.9 Hz), 7.20 (1H, t, J=7.9 Hz), 6.94 (1H, d, J=7.7 Hz), 6.88 (1H, m), 6.85-6.80 (3H, m), 6.77 (1H, ddd, 8.2, 2.6, 1.8 Hz), 6.46 (1H, d, J=11.6 Hz), 6.42 (1H, d, J=15.8 Hz), 6.21 (1H, m), 5.64 (1H, m), 3.813 (3H, s), 3.806 (3H, s), 2.67 (2H, m), 2.54 (4H, m), 2.49 (2H, t, J=7.7 Hz).

### 57. 1,2-Dimethoxy-5-(3′,4′,5′-trimethoxyphenyl)-6,7,8,9-tetrahydro-5H-cyclobenzoheptan-5-ol (57)

3,4,5-Trimethoxybromobenzene was added to anhydrous tetrahydrofuran (60 mL), cooled to −78° C., and stirred for 10 min. n-BuLi (2.5 M, 2.25 mL, 5.6 mmol) was added to the solution and allowed to stir for 1 h. Dimethoxybenzosuberone 19 was dissolved in tetrahydrofuran (5 mL) and slowly added to the solution containing the 3,4,5-trimethoxyphenyl-lithium intermediate. The solution was stirred overnight and allowed to slowly warm to room temperature. On completion, deionized water (10 mL) was added to the solution then the organic solvent was evaporated under reduced pressure. The aqueous reaction mixture was extracted with ethyl acetate (4×30 mL). The combined organic extracts were washed with brine, dried over Na₂SO₄, and evaporated under reduced pressure. Flash chromatography of the crude product using a prepacked 100 g silica gel column [eluent; solvent A, EtOAc, solvent B, hexanes; gradient 5% A/95% B→7% A/3% B (1 CV), 7% A/93% B 53% A/47% B (11 CV); flow rate 40 mL/min; monitored at λλ254 and 280 nm] afforded the tertiary alcohol intermediate 57 (0.73 g, 1.9 mmol, 39% yield) as a light yellow oil that slowly crystallized, R_{f}=0.11 (70:30, Hexanes:EtOAc).

¹H NMR (CDCl₃, 500 MHz): δ 7.27 (1H, d, J=8.7 Hz), 6.76 (1H, d, J=8.8 Hz), 6.50 (2H, s), 3.88 (3H, s), 3.84 (3H, s), 3.75 (9H, s), 3.25 (1H, dd, J=14.3, 7.7 Hz), 2.56 (1H, ddd, J=14.3, 7.1, 3.0 Hz), 2.35 (1H, t, J=12.7 Hz), 2.15 (1H, s), 2.12 (1H, m), 1.94 (1H, m), 1.78 (2H, m), 1.48 (1H, m). ¹³C NMR (CDCl₃, 125 MHz): δ 153.0, 151.8, 146.3, 141.6, 138.6, 137.2, 135.5, 122.8, 108.8, 104.2, 79.9, 61.0, 60.8, 56.1, 55.6, 41.3, 27.1, 26.3, 25.1.

### 58. 1,2-Dimethoxy-5-(3′,4′,5′-trimethoxyphenyl)-6,7,8,9-tetrahydro-5H-cyclobenzohepta-6-ene (58)

Tertiary alcohol analog 57 (0.61 g, 1.6 mmol) was dissolved in acetic acid (10 mL) and refluxed for 4 h. The solution was cooled and deionized water (30 mL) was added to the solution and extracted with diethyl ether (4×40 mL). The combined organic extracts were washed with sat. NaHCO₃ solution (3×50 mL), washed with brine, dried over Na₂SO₄, and evaporated under reduced pressure. The crude product was purified by flash chromatography using a prepacked 50 g silica gel column [eluent; solvent A, EtOAc, solvent B, hexanes; gradient 5% A/95% B 7% A/93% B (1 CV), 7% A/93% B→60% A/40% B (12 CV), 60% A/40% B (2 CV); flow rate 40 mL/min; monitored at λλ254 and 280 nm]. The purification afforded the benzosuberene analog 58 (0.53 g, 1.4 mmol, 92% yield) as a colorless oil, R_{f}=0.46 (70:30, Hexanes:EtOAc).

¹H NMR (CDCl₃, 500 MHz): δ 6.78 (1H, d, J=8.5 Hz), 7.75 (1H, d, J=8.5 Hz), 6.50 (2H, s), 6.34 (1H, T, J=7.4), 3.88 (3H, s), 3.87 (3H, s), 3.86 (3H, s), 3.80 (3H, s), 2.75 (2H, t, J=7.0), 2.15 (2H, p, J=7.1), 1.96 (2H, q, J=7.2). HRMS, m/z: observed 371.1856 [M+H]⁺, (calcd for 371.1853).

### 59. 1,2-Dihydroxy-6,7,8,9-tetrahydro-5H-cyclobenzoheptan-5-one (59)

6,7-Dimethoxybenzosuberone analog 19 (0.49 g, 2.2 mmol) was added to a 20 mL microwave vial with stir bar. To the vial was added 12 mL of anhydrous dichloromethane, then 8.5 mL of [TMAH][Al₂Cl₇] solution (0.926 M, 7.79 mmol) was added to the solution. The vial was sealed and placed in a microwave reaction chamber for 1 h at 80° C. with 30 sec of pre-stirring. The reaction mixture was then slowly added to 50 mL of H₂O. The aqueous reaction mixture was extracted with EtOAc (4×20 mL). The combined organic layers were washed with brine, dried with Na₂SO₄, evaporated under reduced pressure and purified by flash chromatography with a prepacked 25 g silica gel column [eluents; solvent A, EtOAc, solvent B, hexanes; gradient; 7% A/93% B (1 CV), 7% A/93% B 60% A/40% B (13 CV), 60% A/40% B (3 CV); flow rate 25 mL/min; monitored at A's 254 and 280 nm]. Purification afforded catechol 59 (0.42 g, 2.2 mmol, 98%) as a light brown solid, R_{f}=0.14 (70:30 Hexanes:EtOAc).

¹H NMR (DMSO, 500 MHz): δ 10.00 (1H, s), 8.47 (1H, s), 7.01 (1H, d, J=8.5 Hz), 6.69 (1H, d, J=8.5), 2.90 (2H, m), 2.57 (2H, m), 1.65 (4H, m). ¹³C NMR (DMSO, 125 MHz): δ 203.9, 149.5, 142.4, 131.5, 129.7, 120.6, 113.0, 40.6, 24.6, 23.1, 21.2.

### 60. 1,2-Bis((tert-butyldimethylsilyl)oxy)-6,7,8,9-tetrahydro-5H-cyclobenzoheptan-5-one (60)

Catechol analog 59 (0.10 g, 0.47 mmol) was dissolved in DMF (5 mL). To this solution was added tert-butyldimethylsilylchloride (0.18 g, 1.2 mmol) and diisopropylethylamine (0.35 mL, 2.5 mmol) and allowed to stir for 3 h. Distilled H₂O (5 mL) was added to the solution and the aqueous mixture was extracted with diethyl ether (4×10 mL). The combined organic layers were washed with distilled H₂O, washed with brine, dried over Na₂SO₄, and evaporated under reduced pressure, and the crude product was purified by flash chromatography using a prepacked 10 g silica gel column [eluents; solvent A, EtOAc, solvent B, hexanes; gradient, 2% A/98% B (1 CV), 2% A/98% B→20% A/80% B (13 CV), 20% A/80% B (2 CV); flow rate 25 mL/min; monitored at λλ254 and 280 nm]. Purification yielded di-protected analog 60 as a white solid (0.17 g, 0.43 mmol, 87% yield), R_{f}=0.49 (90:10 Hexanes:EtOAc).

¹H NMR (CDCl₃, 500 MHz): δ 7.30 (1H, d, J=8.3 Hz, H=aryl), 6.76 (1H, J=8.6 Hz, H=aryl), 2.96 (2H, m, H=alkyl), 2.70 (2H, m, H=alkyl), 1.80 (4H, m, H=alkyl), 1.03 (9H, s, H=t-Butyl), 0.96 (9H, s, H-t-Butyl), 0.25 (6H, s, H=Si(CH₃)₂), 0.15 (6H, s, H=Si(CH₃)₂). ¹³C NMR (CDCl₃, 125 MHz): δ 205.0, 150.9, 143.7, 135.2, 133.8, 122.4, 118.3, 40.8, 26.23, 26.17, 25.0, 24.8, 21.6, 18.9, 18.6, −3.4, −3.5.

### 61. 1,2-Bis((tert-butyldimethylsilyl)oxy)-5-(3′,4′,5′-trimethoxyphenyl)-6,7,8,9-tetrahydro-5H-cyclobenzoheptan-5-ol (61)

A solution of 3,4,5-trimethoxyphenylbromide (0.525 g, 2.12 mmol) in THF (22 mL) under N₂ was cooled to −78° C., then n-BuLi (1.1 mL, 2.5 M in hexanes) was added and stirred for 1 h. Ketone 60 (0.731 g, 1.74 mmol) was slowly added to the reaction mixture in anhydrous THF (5 mL) and allowed to warm to ambient temperature overnight with continuous stirring. On completion, the reaction mixture was quenched with H₂O (10 mL), and the solvent was evaporated under reduced pressure. The aqueous phase was extracted using EtOAc (4×20 mL). The combined organic extracts were washed with brine, dried over Na₂SO₄, filtered, evaporated under reduced pressure, and purified by flash chromatography using a prepacked 100 g silica column [eluents; solvent A, EtOAc, solvent B, hexanes; gradient, 5% A/95% B (1 CV), 5% A/95% B→59% A/41% B (8.5 CV); flow rate, 40 mL/min; monitored at λλ254 and 280 nm]. Tertiary alcohol 61 (0.632 g, 1.07 mmol, 62% yield) was obtained as a clear liquid.

¹H NMR (CDCl₃, 500 MHz): δ 7.11 (1H, d, J=8.6 Hz), 6.73 (1H, d, J=8.6 Hz), 6.47 (2H, s), 3.83 (3H, s), 3.74 (6H, s), 3.20 (1H, m), 2.56 (1H, m), 2.15 (3H, m), 1.88 (1H, m), 1.73 (2H, m), 1.40 (1H, m), 1.00 (9H, s), 0.95 (9H, s), 0.243 (3H, s), 0.236 (3H, s), 0.16 (3H, s), 0.10 (3H, s). ¹³C NMR (CDCl₃, 125 MHz): δ 153.3, 146.7, 144.0, 142.2, 139.5, 137.5, 134.3, 120.4, 117.8, 104.5, 80.3, 61.2, 56.3, 41.2, 27.2, 26.7, 26.6, 26.5, 26.2, 19.3, 19.0, −3.02, −3.03, −3.05, −3.2.

### 62. 1,2-Bis[(tert-butyldimethylsilyl)oxy]-5-(3′,4′,5′-trimethoxyphenyl)-6,7,8,9-tetrahydro-5H-cyclobenzoheptan-5-ene (62)

Tertiary alcohol 61 (0.472 g, 0.802 mmol) was dissolved in AcOH (3 mL) and stirred overnight at ambient temperature. The reaction was quenched with H₂O (10 mL). The reaction mixture was then extracted with Et₂O (3×15 mL). The combined organic extracts were washed with sat. NaHCO₃, brine, dried over Na₂SO₄, filtered, evaporated under reduced pressure, and purified by flash chromatography using a prepacked 50 g silica gel column [eluent; solvent A, EtOAc, solvent B, hexanes; gradient 0% A/100% B→2% A/98% B (1 CV), 2% A/98% B 17% A/83% B (8.5 CV); flow rate, 40 mL/min; monitored at λλ254 and 280 nm]. TBS-protected benzylidene analog 62 (0.375 g, 0.656 mmol, 82% yield) was obtained as a clear oil, R_{f}=0.36 (90:10, hexanes:EtOAc).

¹H NMR (CDCl₃, 500 MHz): δ 6.69 (1H, d, J=8.3 Hz), 6.53 (1H, d, J=8.3 Hz), 6.46 (2H, s), 6.33 (1H, t, J=7.2 Hz), 3.85 (3H, s), 3.78 (6H, s), 2.71 (2H, t, J=7.0), 2.10 (2H, p, J=7.0), 1.95 (2H, q, J=7.1), 1.04 (9H, s), 0.96 (9H, s), 0.24 (6H, s), 0.20 (6H, s). ¹³C NMR (CDCl₃, 125 MHz): δ 153.1, 146.3, 143.7, 143.4, 138.9, 137.5, 134.7, 134.6, 127.1, 123.0, 118.2, 105.4, 61.2, 26.3, 34.2, 26.62, 26.60, 25.9, 24.9, 19.2, 19.0, −2.96, −3.03.

### 63. 1,2-Dihydroxy-5-(3′,4′,5′-trimethoxyphenyl)-6,7,8,9-tetrahydro-5H-cyclobenzoheptan-5-ol (63)

TBS-protected analog 62 (0.249 g, 0.436 mmol) was added to a flask containing THF (3 mL). To the solution was added TBAF (1.2 mL, 1 M). The solution was stirred for 3 h at room temperature. The reaction was quenched with H₂O (10 mL) and the organic solvent was removed under reduced pressure. The aqueous phase was then extracted with EtOAc (4×15 mL). The combined organic extracts were washed with brine, dried over Na₂SO₄, filtered, evaporated under reduced pressure, and purified by flash chromatography using a prepacked 25 g silica gel column [eluent; solvent A, EtOAc, solvent B, hexanes; gradient 10% A/90% B 12% A/88% B (1 CV), 12% A/88% B→100% A/0% B (12 CV), 100% A/0% B (2 CV); flow rate, 40 mL/min; monitored at λλ254 and 280 nm]. Catechol analog 63 (0.128 g, 0.373 mmol, 86% yield) was obtained as a white solid.

¹H NMR (CDCl₃, 500 MHz): δ 6.67 (1H, d, J=8.2), 6.51 (1H, d, J=8.3), 6.50 (2H, S), 6.33 (1H, t, J=7.4), 5.29 (1H, s), 5.28 (1H, s), 3.86 (3H, s), 3.79 (6H, s), 2.71 (2H, t, J=6.9), 2.15 (2H, p, J=7.0), 1.96 (2H, q, J=7.2). ¹³C NMR (CDCl₃, 125 MHz): δ 152.8, 142.9, 141.9, 140.8, 138.4, 137.2, 134. 1, 128.5, 127.1, 121.7, 112.3, 105.3, 61.0, 56.1, 33.8, 25.6, 23.8

### 64. 5-(3′,4′,5′-Trimethoxyphenyl)-6,7,8,9-tetrahydro-5H-cyclobenzoheptan-5-ol (64)

A solution of 3,4,5-trimethoxyphenylbromide (0.910 g, 3.68 mmol) in THF (40 mL) under N₂ was cooled to −78° C., then n-BuLi (1.5 mL, 2.5 M in hexanes) was added and stirred for 1 h. 6,7,8,9-tetrahydro-5H-benzo[7]annulen-5-one (commercially available from The Aldrich Chemical Company) (0.536 g, 3.34 mmol) was slowly added to the reaction mixture in anhydrous THF (5 mL) and allowed to warm to ambient temperature overnight with continuous stirring. On completion, the reaction mixture was quenched with H₂O (5 mL), and the solvent was evaporated under reduced pressure. The aqueous phase was extracted using EtOAc (4×15 mL). The combined organic extracts were washed with brine, dried over Na₂SO₄, filtered, evaporated under reduced pressure, and purified by flash chromatography using a prepacked 100 g silica column [eluents; solvent A, EtOAc, solvent B, hexanes; gradient, 5% A/95% B 7% A/93% B (1 CV), 7% A/93% B→60% A/40% B (12.5 CV), 60% A/40% B (1 CV); flow rate, 40 mL/min; monitored at λλ254 and 280 nm]. Tertiary alcohol 64 (0.320 g, 0.794 mmol, 11% yield) was obtained as a clear liquid.

¹H NMR (CDCl₃, 500 MHz): δ 7.57 (1H, dd, J=9.0, 1.7 Hz), 7.22 (2H, pd, J=7.3, 1.82 Hz), 7.12 (1H, dd, J=6.6, 1.2 Hz), 6.48 (2H, s), 3.84 (3H, s), 3.74 (6H, s), 2.74 (1H, dd, J=14.4, 6.8 Hz), 2.63 (1H, m), 2.57 (1H, s), 2.21 (1H, m), 2.14 (1H, m), 1.95 (1H, m) 1.78 (2H, m), 1.54 (1H, m). ¹³C NMR (CDCl₃, 125 MHz): δ 153.0, 145.2, 141.3, 141.1, 137.3, 130.5, 127.6, 127.2, 126.2, 104.4, 80.1, 60.8, 56.1, 41.1, 36.3, 27.3, 26.1.

### 65. 5-(3′,4′,5′-trimethoxyphenyl)-6,7,8,9-tetrahydro-5H-cyclobenzohepta-5-ene (65)

Tertiary alcohol 64 (0.123 g, 0.375 mmol) was dissolved in AcOH (5 mL) and stirred for 12 h. To the reaction was added H₂O (40 mL). The aqueous phase was then extracted with EtOAc (3×15 mL). The combined organic extracts were washed H₂O (3×20 mL), washed with brine, dried over Na₂SO₄, filtered, evaporated under reduced pressure, and purified by flash chromatography using a prepacked 25 g silica gel column [eluent; solvent A, EtOAc, solvent B, hexanes; gradient 0% A/100% B 2% A/98% B (1 CV), 2% A/98% B→20% A/80% B (11.5 CV), 20% A/80% B (2.5 CV); flow rate, 40 mL/min; monitored at A's 254 and 280 nm]. Benzylidene analogue 65 (0.082 g, 0.264 mmol, 70% yield) was obtained as a clear oil.

¹H NMR (CDCl₃, 500 MHz): δ 7.28 (1H, dd, J=7.3, 1.7 Hz), 7.21 (2H, m), 7.05 (1H, dd, J=7.1, 2.0 Hz), 6.49 (2H, s), 6.42 (1H, t, J=7.3 Hz), 3.86 (3H, s), 3.79 (6H, s), 2.67 (2H, t, J=7.1 Hz), 2.19 (2H, p, J=7.1 Hz), 1.97 (2H, q, J=7.2 Hz). ¹³C NMR (CDCl₃, 125 MHz): δ 152.9, 143.0, 142.2, 140.0, 138.2, 137.4, 129.4, 128.6, 128.1, 127.1, 125.8, 105.3, 60.9, 56.1, 35.1, 32.4, 25.4.

### 66. (E)/(Z) 6-(2′,3′-Dimethoxyphenyl)hex-5-enoic acid (66)

To a solution of THF (500 mL) was added (4-carboxybutyl)triphenylphosphonium bromide (28.7 g, 64.9 mmol) and potassium tert-butoxide (15.9 g, 141.7 mmol). The solution was allowed to stir for 1 h at ambient temperature. 2,3-Dimethoxybenzaldehyde (10.6 g, 63.9 mmol) was dissolved into THF (10 mL) then added drop-wise to the reaction mixture, which was allowed to stir for 12 h at ambient temperature. The solution was quenched with 2M HCl (25 mL), and the organic solvent was evaporated under reduced pressure. The aqueous phase was extracted with EtOAc (4×50 mL), washed with brine, dried over Na₂SO₄, filtered, evaporated under reduced pressure, and purified by flash chromatography with a prepacked 340 g silica gel column [eluents; solvent A, EtOAc, solvent B, hexanes; gradient, 5% A/95% B→7% A/93% B (1 CV), 7% A/93% B 60% A/40% B (13 CV), 60% A/40% B (2 CV); flow rate, 40 mL/min; monitored at λλ254 and 280 nm]. Hexenoic acid analog 66 (6.21 g, 24.8 mmol, 46% yield) was obtained as a mixture of E and Z isomers and was a yellow oil.

### 67. 6-(2′,3′-Dimethoxyphenyl)hexanoic acid (67)

Hexenoic acid analog 66 (6.21 g, 24.8 mmol) under N₂ was added to anhydrous MeOH (75 mL) under N₂. To this solution was added 10% Pd/C (0.431 g) and stirred under H₂ (in balloons) then stirred for 12 h. The reaction mixture was filtered through CELITE (diatomaceous earth), washed with EtOAc (4×50 mL), evaporated under reduced pressure, and purified by flash chromatography using 20% EtOAc/80% hexanes as eluent. Hexanoic acid analog 67 (6.26 g, 24.8 mmol, 100% yield) was obtained as a clear oil.

¹H NMR (CDCl₃, 500 MHz): δ 6.97 (1H, m), 6.76 (2H, m), 3.85 (3H, s), 3.81 (3H, s), 2.62 (2H, m), 2.36 (2H, m), 1.64 (4H, m), 1.41 (2H, m).

### 68. 1,2-dimethoxy-7,8,9,10-tetrahydrocyclobenzooctan-5-one (68)

To a flask containing hexanoic acid analog 67 (6.09 g, 24.1 mmol) was added 250 mL of DCM and cooled to 0° C. Eaton's reagent (50 mL, 7.7% P₂O₅ in CH₃SO₃H) was then added to the solution. The solution was stirred vigorously and allowed to slowly warm to ambient temperature over 12 h. The solution was poured over ice, which was allowed to melt, then slowly neutralized with NaHCO₃ (aq.). The aqueous phase was extracted with Et₂O (4×50 mL). The combined organic extracts were washed with brine, dried over Na₂SO₄, filtered, evaporated under reduced pressure, and purified using flash chromatography with a prepacked 100 g silica column [eluents; solvent A, EtOAc, solvent B, hexanes; gradient, 5% A/95% B 7% A/93% B (1 CV), 7% A/93% B→37% A/63% B (8 CV); flow rate, 40 mL/min; monitored at λλ254 and 280 nm]. Ketone 68 (1.26 g, 5.36 mmol, 22% yield) was obtained as a clear oil.

¹H NMR (CDCl₃, 500 MHz): δ 7.55 (1H, d, J=8.8 Hz), 6.84 (1H, d, J=8.8 Hz), 3.90 (3H, s), 3.80 (3H, s), 3.13 (2H, m), 1.81 (4H, m), 1.50 (2H, m). ¹³C NMR (CDCl₃, 125 MHz): δ 205.4, 155.6, 146.5, 134.9, 133.6, 124.9, 109.6, 60.8, 55.7, 44.0, 27.1, 25.5, 24.8, 24.2.

### 69. 1,2-Dihydroxy-7,8,9,10-tetrahydrocyclobenzooctan-5-one (69)

6,7-Dimethoxybenzocyclooctanone analog 68 (0.276 g, 1.18 mmol) was added to 20 mL microwave vial with stir bar. To the vial was added 12 mL of anhydrous dichloromethane, then [TMAH][Al₂Cl₇] solution (4.8 mL, 0.496 M, 2.38 mmol) was added to the solution. The vial was sealed and placed in a microwave reaction chamber for 1 h at 80° C. with 30 sec of pre-stirring. The reaction mixture was then slowly added to 50 mL of DI H₂O. The aqueous reaction mixture was extracted with EtOAc (4×20 mL). The combined organic layers were washed with brine, dried with Na₂SO₄, evaporated under reduced pressure and purified by flash chromatography with a prepacked 25 g silica gel column [eluents; solvent A, EtOAc, solvent B, hexanes; gradient; 5% A/95% B→7% A/93% B (1 CV), 7% A/93% B→60% A/40% B (10 CV), 60% A/40% B (2 CV); flow rate 25 mL/min; monitored at λλ254 and 280 nm]. Purification afforded catechol 69 (0.141 g, 0.686 mmol, 58%) as a light tan solid, R_{f}=0.16 (70:30 Hexanes:EtOAc).

¹H NMR (CDCl₃, 500 MHz): δ 7.16 (1H, d, J=8.2 Hz), 6.75 (1H, d, J=8.4), 3.06 (2H, m), 2.91 (2H, m), 1.82 (4H, m), 1.56 (2H, m).

### 70. 1,2-Bis((tert-butyldimethylsilyl)oxy)-7,8,9,10-tetrahydrocyclobenzooctan-5-one (70)

Catechol analog 69 (0.141 g, 0.686 mmol) was dissolved in DMF (2 mL). To this solution was added tert-butyldimethylsilylchloride (0.280 g, 1.86 mmol) and diisopropylethylamine (0.50 mL, 3.6 mmol) and allowed to stir for 12 h. Water (5 mL) was added to the solution and the aqueous mixture was extracted with diethyl ether (4×10 mL). The combined organic layers were washed with distilled H₂O, washed with brine, dried over Na₂SO₄, and evaporated under reduced pressure, and the crude product was purified by flash chromatography using a prepacked 10 g silica gel column [eluents; solvent A, EtOAc, solvent B, hexanes; gradient, 5% A/95% B→7% A/93% B (1 CV), 7% A/93% B→40% A/60% B (8 CV); flow rate 40 mL/min; monitored at λλ254 and 280 nm]. Purification yielded di-protected analog 70 as a white solid (0.228 g, 0.524 mmol, 76% yield).

¹H NMR (CDCl₃, 500 MHz): δ 7.06 (1H, d, J=8.5 Hz), 6.60 (1H, d, J=8.5 Hz), 2.97 (2H, t, J=6.2 Hz,), 2.84 (2H, t, J=6.5 Hz), 1.76 (4H, m), 1.51 (2H, m), 1.02 (9H, s), 0.95 (9H, s), 0.24 (6H, s), 0.16 (6H, s).

### 71. 9-(4′-Hydroxy-3′,5′-dimethoxyphenyl)-3-methoxy-6,7-dihydro-5H-benzo[7]annulen-4-ol (71)

3,4-Dimethoxy-9-(3,4,5-trimethoxyphenyl)-6,7-dihydro-5H-benzo[7]annulene analog 58 (0.336 g, 0.906 mmol) was added to 5 mL microwave vial with stir bar. To the vial was added [TMAH][Al₂Cl₇] solution (2.0 mL, 1.06 M, 2.12 mmol). The vial was sealed and placed in a microwave reaction chamber for 1 h at 80° C. with 30 sec of pre-stirring. The reaction mixture was then slowly added to H₂O (20 mL). The aqueous reaction mixture was extracted with EtOAc (4×25 mL). The combined organic layers were washed with brine, dried with Na₂SO₄, evaporated under reduced pressure and purified by flash chromatography with a prepacked 100 g silica gel column [eluents; solvent A, EtOAc, solvent B, hexanes; gradient; 5% A/95% B→7% A/93% B (1 CV), 7% A/93% b 60% A/40% B (12 CV); flow rate 20 mL/min; monitored at A's 254 and 280 nm]. Di-phenol analogue 71 (0.041 g, 0.121 mmol, 13% yield) was obtained as a red solid, R_{f}=0.15 (70:30 Hexanes:EtOAc). Two additional compounds were also elucidated from this reaction. One compound being 3,4-dimethoxy-9-(3′,5′-dimethoxy-4′-hydroxy phenyl)-6,7-dihydro-5H-benzo[7]annulene, the other being 3-methoxy-4-hydroxy-9-(3′,4′-dihydroxy-5′-methoxyphenyl)-6,7-dihydro-5H-benzo[7]annulene.

¹H NMR (CDCl₃, 500 MHz): δ 6.70 (1H, d, J=8.4 Hz), 6.56 (1H, d, J=8.4 Hz), 6.51 (2 H, s), 6.29 (1H, t, J=7.4 Hz), 5.74 (1H, s), 5.48 (1H, s), 3.91 (3H, s), 3.83 (6H, s), 2.74 (2H, t, J=7.0 Hz), 2.14 (2H, p, J=7.3 Hz), 1.95 (2H, q, J=7.2 Hz).

### 72. 2-Methoxy-5-(3,4,5-trimethoxyphenyl)-6,7,8,9-tetrahydro-5H-benzo[7]annulen-1-ol (72)

Benzosuberene analogue 23 (0.102 g, 0.286 mmol) under N₂ was added to MeOH (3 mL) under N₂. To this solution was added 10% Pd/C (cat. amount) and stirred under H₂ (in balloons) then stirred for 12 h. The reaction mixture was filtered through CELITE, washed with EtOAc (4×50 mL), evaporated under reduced pressure, and purified by flash chromatography with a prepacked 100 g silica gel column [eluents; solvent A, EtOAc, solvent B, hexanes; gradient; 5% A/95% B (1 CV), 5% A/95% b 56% A/44% B (11 CV); flow rate 35 mL/min; monitored at A's 254 and 280 nm]. Annulene analogue 72 (0.0327 g, 0.091 mmol, 32% yield) was obtained as white solid.

### 73. 6-methoxy-3-(3′,4′,5′-trimethoxyphenyl)-1H-inden-7-ol (73)

### 74. 2-methoxy-5-(3′,4′,5′-trimethoxyphenyl)-7,8-dihydronaphthalen-1-ol (74)

### 75. 3-methoxy-5-(3′,4′,5′-trimethoxyphenyl)-7,8-dihydronaphthalene-1,2-diol (75)

Compounds 73, 74, and 75 were synthesized consistent with the methods described above.

### B. Alternate Synthetic Method

### 1. 1-Nitro-2-Methoxy-6,7,8,9-tetrahydro-5H-cyclobenzoheptan-5-one (3)

To a solution of benzosuberone 48 (0.533 g, 2.80 mmol) in acetic anhydride (5 mL) cooled to −78° C., a solution (1:1) of HNO₃ and acetic acid (1.00 mL) was added dropwise, while stirring the solution. The reaction mixture was allowed to stir for 3 h and then ice cold water was added to quench the reaction. The mixture was stirred vigorously for 2 min and the organic layer was separated. Aqueous layer was extracted with EtOAc (2×25 mL). Combined organic phase was washed with brine, dried over MgSO₄, filtered and the solvents evaporated on a rotavapor. Flash chromatography of the crude using a prepacked 50 g silica column; Eluents; solvent A, EtOAc, solvent B, hexanes; gradient, 20% A/80% B over 3.18 min (1 CV), 20% A/80% B 80% A/20% B over 33.0 min (10 CV), 80% A/20% B over 6.36 min (2 CV); flow rate 40.0 mL/min; monitored at λλ254 and 280 nm; afforded 1-nitrobenzosuberone 3 (0.201 g, 0.854 mmol, 30%) and 3-nitro benzosuberone 76 (0.268 g, 1.14 mmol, 41%).

¹H NMR (1-nitro benzosuberone 3) (500 MHz, CDCl₃) δ 7.84 (1H, d, J=8.8 Hz, H-4), 6.97 (1H, d, J=8.8 Hz, H-3), 3.94 (3H, s, OCH₃-2), 2.79 (2H, dd, J=6.5 Hz, H-9), 2.72 (2H, dd, J=6.0 Hz, H-6), 1.92 (2H, m, J=6.5 Hz, H-8), 1.82 (2H, m, J=6.0 Hz, H-7); ¹³C NMR (126 MHz, CDCl₃) δ 203.1 (C, C-5), 153.3 (C, C-2), 141.4 (C, C-1), 134.0 (C, C-1a), 132.2 (C, C-4-a), 131.9 (C, C-4), 110.3 (C, C-3), 56.6 (CH₃, OCH₃-2), 40.3 (C, C-6), 26.2 (C, C-9), 24.4 (C, C-8), 20.2 (C, C-7); Anal., Calcd for C₁₂H₁₃NO₄: C, 61.27; H, 5.57; N, 5.82. Found: C, 61.17; H, 5.55; N, 5.82; HRMS, m/z: observed 236.0919 [M+1]⁺, (calcd for C₁₂H₁₄NO₄⁺, 236.0917).

¹H NMR (3-nitro benzosuberone 76) (500 MHz, CDCl₃) δ 8.32 (1H, s, H-4), 6.88 (1H, s, H-1), 4.01 (3H, s, OCH₃-2), 2.99 (2H, dd, J=6.6, 6.3 Hz, H-9), 2.76 (2H, dd, J=6.1, 4.1 Hz, H-6), 1.94 (2H, m, J=6.6, 6.3 Hz, H-8), 1.84 (2H, m, J=6.1, 4.1 Hz, H-7); ¹³C NMR (126 MHz, CDCl₃) δ 202.0 (C, C-5), 155.1 (C, C-2), 148.8 (C, C-1a), 138.2 (C, C-3), 131.0 (C, C-4-a), 127.3 (C, C-4), 114.1 (C, C-1), 56.7 (CH₃, OCH₃-2), 40.4 (C, C-6), 33.1 (C, C-9), 24.7 (C, C-8), 20.3 (C, C-7); HRMS, m/z: observed 236.0921 [M+1]⁺, (calcd for C₁₂H₁₄NO₄⁺, 236.0917).

### 2. 1-Nitro-2-Methoxy-5-(3′,4′,5′-trimethoxyphenyl)-6,7,8,9-tetrahydro-5H-cyclobenzoheptan-5-ol (4)

To a solution of 3,4,5-trimethoxyphenylbromide (1.00 g, 4.04 mmol) in anhydrous THF (50 mL) at −78° C., n-BuLi (1.60 mL, 2.5 M) was added and the reaction stirred for 30 min. 1-Nitro-benzosuberone 3 (0.20 g, 0.85 mmol) in 5 mL THF was added using a dropping funnel over a period of 15 min. The reaction mixture was stirred overnight and allowed to warm to ambient temperature. The reaction mixture was quenched with H₂O (10 mL), and extracted with EtOAc (2×25 mL). The combined organic phase was washed with brine, dried over MgSO₄, filtered, and evaporated under reduced pressure. Flash chromatography of the crude product using a prepacked 25 g silica column [eluents; solvent A, EtOAc, solvent B, hexanes; gradient, 20% A/80% B over 1.39 min (1 CV), 20% A/80% B→80% A/20% B over 16.3 min (10 CV), 80% A/20% B over 5.18 min (2 CV); flow rate 25.0 mL/min; monitored at λλ254 and 280 nm] afforded alcohol 4 (0.050 g, 0.12 mmol, 15% yield) as a white solid.

¹H NMR (500 MHz, CDCl₃) δ 7.68 (1H, d, J=8.9 Hz, H-4), 6.88 (1H, d, J=8.9 Hz, H-3), 6.46 (2H, S, H-2′, -6′), 3.89 (3H, s, OCH₃-2), 3.84 (3H, s, OCH₃-4′), 3.76 (3H, s, OCH₃-3′, -5′), 2.62-2.58 (1H, m, H-9), 2.61-2.56 (1H, m, H-6), 2.42-2.36 (1H, m, H-9), 2.15-2.09 (1H, m, H-6), 1.96-1.93 (1H, m, H-8), 1.85-1.80 (1H, m, H-8), 1.80-1.72 (1H, m, H-7), 1.55-1.48 (1H, m, H-7). ¹³C NMR (126 MHz, CDCl₃) δ 153.3 (C, C-3′, -5′), 149.3 (C, C-2), 142.4 (C, C-1), 140.4 (C, C-1′), 138.5 (C, C-4-a), 137.6 (C, C-4′), 133.5 (C, C-1a), 129.3 (CH, C-4), 109.2 (CH, C-3), 104.0 (CH, C-2′, -6′), 79.7 (C, C-5), 60.9 (CH₃, OCH₃-4′), 56.23 (CH₃, OCH₃-2), 56.18 (CH₃, OCH₃-3′, -5′), 40.8 (CH₂, CH₂-6), 28.6 (CH₂, CH₂-9), 26.3 (CH₂, CH₂-7), 25.9 (CH₂, CH₂-8). HRMS, m/z: observed 426.1523 [M+Na]⁺, (calcd for C₂₁H₂₅NO₇Na⁺, 426.1523).

### 3. 1-Nitro-2-Methoxy-5-(3′,4′,5′-trimethoxyphenyl)-6,7,8,9-tetrahydro-5H-cyclobenzohepta-6-ene (5)

A solution of 4 (0.19 g, 0.47 mmol) in AcOH (6 mL) and H₂O (4 mL) was heated to reflux at 160° C. for 12 h. The reaction mixture was cooled and the aqueous solvents evaporated over a rotavapor to obtain a crude product. Flash chromatography of the crude product using a prepacked 25 g silica column [eluents; solvent A, EtOAc, solvent B, hexanes; gradient, 20% A/80% B over 1.39 min (1 CV), 20% A/80% B→80% A/20% B over 16.3 min (10 CV), 80% A/20% B over 5.18 min (2 CV); flow rate 25.0 mL/min; monitored at λλ254 and 280 nm] afforded benzosuberene 5 (0.104 g, 0.27 mmol, 57% yield) as a white solid.

¹H NMR (500 MHz, CDCl₃) δ 7.12 (1H, d, J=9.0 Hz, H-4), 6.87 (1H, d, J=9.0 Hz, H-3), 6.45 (2H, S, H-2′, -6′), 6.43 (2H, t, J=7.5 Hz, H-6), 3.91 (3H, s, OCH₃-3), 3.87 (3H, s, OCH₃-4′), 3.82 (3H, s, OCH₃-3′, -5′), 2.56 (2H, t, J=7.0 Hz, H-9), 2.22 (2H, p, J=7.0 Hz, H-8), 2.00 (2H, dt, J=7.0, 7.5 Hz, H-7). ¹³C NMR (126 MHz, CDCl₃) δ 153.1 (C, C-3′, -5′), 149.3 (C, C-3), 141.5 (C, C-4), 141.5 (C, C-5), 137.7 (C, C-4′), 137.3 (C, C-1′), 134.9 (C, C-4-a), 133.7 (C, C-1 a), 131.8 (CH, C-1), 128.6 (CH, C-8), 109.6 (CH, C-2), 105.1 (CH, C-2′, -6′), 60.9 (CH₃, OCH₃-4′), 56.3 (CH₃, OCH₃-3′, -5′), 56.2 (CH₃, OCH₃-3), 34.7 (CH₂, CH₂-6), 27.3 (CH₂, CH₂-5), 25.2 (CH₂, CH₂-7). Anal., Calcd for C₂₁H₂₃NO₆: C, 65.44; H, 6.02; N, 3.63, 0, 24.91. Found: C, 65.41; H, 6.19; N, 3.58. HRMS, m/z: observed 386.1600 [M+1]+, (calcd for C₂₁H₂₄ NO₆⁺, 386.1598).

### 4. 2-Methoxy-5-(3′,4′,5′-trimethoxyphenyl)-6,7,8,9-tetrahydro-5H-cyclobenzohepta-6-ene-1-amine (6)

A suspension of benzosuberene 5 (0.049 g, 0.217 mmol) and Zinc (0.43 g) in AcOH (10 mL) was stirred for 2 h. The reaction mixture was filtered and the aqueous solvents evaporated over a rotavapor to obtain a crude product. Flash chromatography of the crude product using a prepacked 10 g silica column [eluents; solvent A, EtOAc, solvent B, hexanes; gradient, 30% A/70% B over 1.39 min (1 CV), 30% A/70% B→30% A/70% B over 16.3 min (10 CV), 80% A/20% B over 5.18 min (2 CV); flow rate 12.0 mL/min; monitored at λλ254 and 280 nm] afforded amine benzosuberene 6 (0.040 g, 0.113 mmol, 88% yield) as a white solid.

¹H NMR (500 MHz, CDCl₃) δ 6.67 (1H, d, J=8.4 Hz, H-3), 6.52 (2H, S, H-2′, -6′), 6.49 (1H, d, J=8.4 Hz, H-4), 6.30 (2H, t, J=7.3 Hz, H-6), 3.88 (3H, s, OCH₃-2), 3.86 (3H, s, OCH₃-4′), 3.80 (3H, s, OCH₃-3′, -5′), 2.59 (2H, t, J=7.0 Hz, H-9), 2.12 (2H, p, J=7.0 Hz, H-8), 1.95 (2H, dt, J=7.0, 7.3 Hz, H-7). ¹³C NMR (126 MHz, CDCl₃) δ 152.8 (C, C-3′, -5′), 146.3 (C, C-2), 143.5 (C, C-5), 138.5 (C, C-1′), 137.2 (C, C-4′), 133.5 (C, C-4-a), 132.4 (C, C-1), 126.8 (C, C-1a), 126.3 (CH, C-6), 119.8 (CH, C-4), 107.5 (CH, C-3), 105.2 (CH, C-2′, -6′), 60.9 (CH₃, OCH₃-4′), 56.1 (CH₃, OCH₃-3′, -5′), 55.5 (CH₃, OCH₃-2), 33.2 (CH₂, CH₂-8), 25.6 (CH₂, CH₂-7), 25.3 (CH₂, CH₂-9). HRMS, m/z: observed 356.1862 [M+1]⁺, (calcd for C₂₁H₂₆NO₄⁺, 356.1856).

### C. Effects on Tubulin Polymerization

Bovine brain tubulin was purified using methods previously described by Hamel (Cell Biochem. Biophys. 38:1-21 (2003)). The effect of compounds on tubulin assembly in vitro was determined by using a series of concentrations that were pre-incubated with 10 μM tubulin (1.0 mg/mL) in glutamate buffer at 30° C., followed by cooling to 0° C. After GTP was added, the samples were mixed and transferred to cuvettes at 0° C. in a recording spectrophotometer and warmed to 30° C. to initiate polymerization. Tubulin assembly was observed turbidimetrically at 350 nm. Tubulin disassembly was confirmed by cooling to 0° C. The calculated compound concentration that inhibited tubulin assembly by 50% after a 20 min incubation was defined as the IC₅₀ value.

Benzosuberene analogs 5, 6 and 23 were found to be potent inhibitors of tubulin assembly comparable to CA1, CA4, and.

TABLE 1
Inhibition of tubulin polymerization

| | Inhibition of tubulin |
|---|---|
| | polymerization |
| Compound | IC₅₀ (μM) |
| CA1 | 1.9 |
| CA4 | 1.2 |
| 5 | 2.5 |
| 6 | 1.2 |
| 23 | 1.7 |

### D. Cell Lines, and Sulforhodamine B (SRB) Assay

Cancer cell lines were obtained from ATCC (DU-145 (prostate), SK-OV-3 (ovarian), and NCI-H460 (lung)) and maintained according to recommended conditions. Media was enriched with the recommended concentration of fetal bovine serum, as well as gentamicin and amphotericin B. The National Cancer Institute's standard SRB assay assessed cancer cell line growth inhibition, as previously described as the GI₅₀, or the drug concentrations calculated to cause a 50% reduction in net protein increase relative to untreated cells (Vichai and Kirtikara, Nat. Protocols 1:1112-1116 (2006); Monks, et al., J. Natl. Cancer Inst. 83″757-766 (1991); Sites, et al., J. Nat. Prod. 71:313-320 (2008)). Results reported are averages of at least three separate experiments, each of which was carried out in triplicate.

The amino benzosuberene analog 6 demonstrated remarkable cytotoxicity against ovarian cancer with a GI₅₀ value of 32.9 μM. In addition, the compound was strongly cytotoxic against the non-small cell lung and prostate cell lines. While somewhat less active than the closely related benzosuberene phenol 23, the amino derivative 6 was more active against each cell line than the natural products CA4 and CA1. The nitro benzosuberene analog 5 was significantly less cytotoxic than any of the comparison compounds (Table 2).

TABLE 2
Cytotoxicity studies against human cancer cell lines

| cpd | SK-OV-3 | NCI-H460 | DU-145 |
|---|---|---|---|
| 5 | 0.152 | 0.666 | 0.211 |
| 6 | 0.0000329 | 0.00469 | 0.00111 |
| 13 | 22.7 | 20.9 | 20.5 |
| 16 | 7.13 | 18.0 | 15.7 |
| 23 | 0.0000242 | 0.00000192 | 0.00000242 |
| 24 | 0.00594 | 0.0266 | 0.00966 |
| 36 | 18.1 | 25.9 | 16.3 |
| 41 | 0.00000724 | 0.00400 | 0.00287 |
| 46 | 0.0399 | 0.0832 | 0.108 |
| 50 | 0.0366 | 0.0510 | 0.0261 |
| 58 | 0.641 | 0.479 | 0.516 |
| 63 | 0.341 | 0.410 | 0.783 |
| 65 | 19.1 | 32.5 | 22.0 |
| 71 | 0.378 | 0.439 | 0.579 |

We claim:

1. A process of making a compound of formula I, 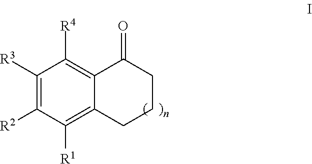
wherein
R¹, R², R³, and R⁴ are selected from the group consisting of hydrogen, halogen, C₁-C₆ alkyl, C₁-C₆ alkoxy, hydroxy, amino, nitro, and acylamino; and
n is an integer selected from 0, 1, 2 and 3;
said process comprising:
reacting a benzaldehyde of formula A-1 with a Wittig reagent having the formula Ph₃P⁺(CH₂)₍ₙ₊₁₎COOH; 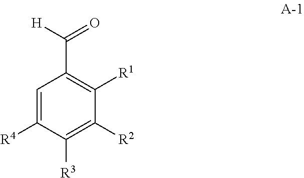
reducing the double bond formed in the Wittig reaction; and
effecting ring closure to form a compound of formula I.

2. The process of claim 1, wherein n is 2.

3. The process of claim 1, wherein ring closure is effected with Eaton's reagent.

4. A process of making a compound of formula II 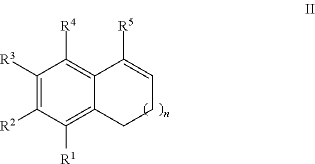
wherein
R¹, R², R³, and R⁴ are selected from the group consisting of hydrogen, halogen, C₁-C₆ alkyl, C₁-C₆ alkoxy, hydroxy, amino, and acylamino;
R⁵ is selected from the group consisting of unsubstituted aryl, substituted aryl, unsubstituted arylcarbonyl and substituted arylcarbonyl; and
n is an integer selected from 0, 1, 2 and 3;
said process comprising:
reacting a benzaldehyde of formula A-1 with a Wittig reagent having the formula Ph₃P⁺(CH₂)₍ₙ₊₁₎COOH; 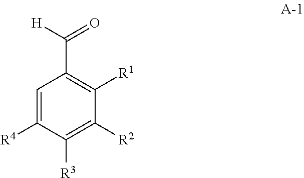
reducing the double bond formed in the Wittig reaction;
effecting ring closure to form a benzoannulenone; and
adding an R⁵ moiety at the ketone position of the benzoannulenone.

5. The process of claim 4, wherein R⁵ is a substituted or unsubstituted aryl, and wherein adding the R⁵ moiety at the ketone position of the benzoannulenone comprises:
reacting the benzoannulenone directly with R⁵—Li to form a tertiary alcohol; and
eliminating the tertiary alcohol to form the compound of formula II.

6. The process of claim 4, wherein R⁵ is a substituted or unsubstituted arylcarbonyl, and wherein adding the R⁵ moiety at the ketone position of the benzoannulenone comprises:
converting the ketone of the benzoannulenone to a vinyl-lithium intermediate;
reacting the intermediate with an electrophile to form an alcohol; and
oxidizing the resultant alcohol to form the compound of Formula II.
